(19) United States
(12) Patent Application Publication
(10) Pub. No.: US 2024/0285634 A1
(43) Pub. Date: Aug. 29, 2024

(54) COMPOUND AND METHOD FOR TREATING CHEMOTHERAPY-ASSOCIATED GASTROINTESTINAL SIDE EFFECTS

(71) Applicant: ONQUALITY PHARMACEUTICALS CHINA LTD., Shanghai (CN)

(72) Inventors: Wenxi LI, Shanghai (CN); Shilan LIU, Shanghai (CN); Zhaoyu WU, Shanghai (CN); Shiyi ZHANG, Shanghai (CN)

(73) Assignee: ONQUALITY PHARMACEUTICALS CHINA LTD., Shanghai (CN)

(21) Appl. No.: 18/568,477

(22) PCT Filed: Jun. 9, 2022
(86) PCT No.: PCT/CN2022/097793

§ 371 Date: Dec. 8, 2023

(30) Foreign Application Priority Data
Jun. 10, 2021 (CN) 202110649083.7

(51) Int. Cl. A61K 31/519; A61K 45/06; A61P 1/10

(52) U.S. Cl. CPC A61K 31/519; A61K 45/06; A61P 1/10

(57) ABSTRACT

A compound and method may treat chemotherapy-associated gastrointestinal side effects. A compound of formula (I) or a pharmaceutically acceptable salt thereof in the preparation of a medicament:

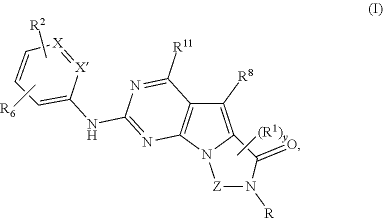

may be used for preventing, alleviating, and/or treating gastrointestinal side effects associated with chemotherapy in a subject. A method may use such a compound to treat chemotherapy-associated gastrointestinal side effects.

### TECHNICAL FIELD

The present application relates to the field of biomedicine, and particularly to a method of preventing, relieving and/or treating chemotherapy-associated gastrointestinal side effects.

### BACKGROUND

Tumor chemotherapy is one of the most commonly used means for treating tumors clinically, which mainly utilizes a chemical medicament (a chemotherapeutic agent) to prevent the proliferation, infiltration, and metastasis of cancer cells, eventually killing cancer cells. However, the administration of a chemotherapeutic agent can not only kill rapidly growing and isolated tumor cells, it may also kill normal cells and immune cells at the same time, thus causing severe side effects, including nausea, vomiting, anemia, inflammation, etc. Such side effects may result in withdrawal of the chemotherapeutic agent or dosage reduction, greatly affecting the therapeutic effect on the tumor of patients, and impairing the quality of life for patients, and endanging the patient's life in severe cases.

No successful therapeutic regimen has been available yet in the prior art for controlling the side effects associated with the administration of a chemotherapeutic agent. Therefore, there is an urgent need for therapeutic regimens that can successfully control these side effects.

### SUMMARY OF THE INVENTION

The present application provides a method of preventing, relieving and/or treating chemotherapy-associated gastrointestinal side effects (e.g., diarrhea, enteritis or constipation) in a subject, comprising administering a compound as shown in Formula I or a pharmaceutically acceptable salt thereof to the subject. The compound as shown in Formula I of the present application or the pharmaceutically acceptable salt thereof is capable of effectively preventing, relieving and/or treating chemotherapy-associated gastrointestinal side effects.

In one aspect, the present application provides a use of a compound as shown in Formula I or a pharmaceutically acceptable salt thereof in preparing a medicament, which is used for preventing, relieving and/or treating chemotherapy-associated gastrointestinal side effects in a subject:

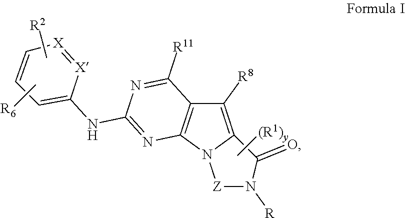

wherein,
Z is —(CH₂)ₓ—, wherein X is 1, 2, 3 or 4, or —O—(CH₂)_{z}—, wherein z is 2, 3 or 4;

X and X′ are each independently CH or N;
R, R⁸ and R¹¹ are each independently H, C₁-C₃ alkyl or haloalkyl, cycloalkyl or cycloalkyl containing one or more heteroatoms selected from N, O or S, -(alkylene)ₘ-C₃-C₈ cycloalky), -(alkylene)ₘ-aryl, -(alkylene)ₘ-heterocyclyl, -(alkylene)ₘ-heteroaryl, -(alkylene)ₘ-NR³R⁴, -(alkylene)ₘ-C(O)—NR³R⁴, -(alkylene)ₘ-O—R⁵, -(alkylene)ₘ-S(O)—R⁵, or -(alkylene)ₘ-S(O)ₙ—NR³R⁴, any of which may be optionally independently substituted with one or more R groups as allowed by valance, and wherein two R^{x} groups bound to the same or adjacent atoms may optionally combine to form a ring, and wherein m is 0 or 1, n is 0, 1 or 2;
R¹ is independently aryl, alkyl, cycloalkyl or haloalkyl, wherein each of the alkyl, cycloalkyl and haloalkyl optionally comprises O or N heteroatoms in place of a carbon in the chain and two R¹'s on adjacent ring atoms or on the same ring atom together with the ring atom(s) to which they are attached optionally form a 3-8-membered cycle;
y is 0, 1, 2, 3 or 4:

R² is -(alkylene)ₘ-heterocyclyl, -(alkylene)ₘ-heteroaryl, -(alkylene)ₘ-NR³R⁴, -(alkylene)ₘ-C(O)—NR³R⁴, -(alkylene)ₘ-C(O)—O-alkyl; -(alkylene)ₘ-O—R⁵, -(alkylene)ₘ-S(O)ₙ—R⁵ or -(alkylene)ₘ-S(O)ₙ— NR³R⁴, any of which may be optionally independently substituted with one or more R^{x} groups as allowed by valance, and wherein two R^{x} groups bound to the same or adjacent atoms may optionally combine to form a ring, and wherein m is 0 or 1, n is 0, 1 or 2;
R³ and R⁴ at each occurrence are independently:
(i) hydrogen or
(ii) alkyl, cycloalkyl, heterocyclyl, aryl, heteroaryl, cycloalkylalkyl, heterocycloalkyl, arylalkyl or heteroarylalkyl, any of which may be optionally independently substituted with one or more R^{x} groups as allowed by valance, and wherein two R^{x} groups bound to the same or adjacent atoms may optionally combine to form a ring; or R³ and R⁴ together with the nitrogen atom to which they are attached may combine to form a heterocyclic ring optionally independently substituted with one or more R^{x} groups as allowed by valance, and wherein two R^{x} groups bound to the same or adjacent atoms may optionally combine to form a ring;
R⁵ and R⁵* at each occurrence is:
(i) hydrogen or
(ii) alkyl, alkenyl, alkynyl, cycloalkyl, heterocyclyl, aryl, heteroaryl, cycloalkylalkyl, heterocycloalkyl, arylalkyl or heteroarylalkyl, any of which may be optionally independently substituted with one or more RY groups as allowed by valance:

R^{x} at each occurrence is independently halo, cyano, nitro, oxo, alkyl, haloalkyl, alkenyl, alkynyl, cycloalkyl, cycloalkenyl, heterocyclyl, aryl, heteroaryl, arylalkyl, heteroarylalkyl, cycloalkylalkyl, heterocycloalkyl, -(alkylene)ₘ-OR⁵, -(alkylene)ₘ-O-alkylene-OR⁵, -(alkylene)ₘ-S(O)ₙ—R⁵, -(alkylene)ₘ-NR³R⁴, -(alkylene)ₘ-CN, -(alkylene)ₘ-C(O)—R⁵, -(alkylene)ₘ-C(S)—R⁵, -(alkylene)ₘ-C(O)—OR⁵, -(alkylene)ₘ-O—C(O)—R⁵, -(alkylene)ₘ-C(S)—OR⁵, -(alkylene)ₘ-C(O)-(alkylene)ₘ-NR³R⁴, -(alkylene)ₘ-C(S)—NR³R⁴, -(alkylene)ₘ-N(R³)—C(O)—NR³R⁴, -(alkylene)ₘ-N(R³)—C(S)—NR³R⁴, -(alkylene)ₘ-N(R³)—C(O)—R⁵, -(alkylene)ₘ-N(R³)—C(S)—R⁵, -(alkylene)ₘ-O—C(O)—NR³R⁴, -(alkylene)ₘ-O—C(S)—NR³R⁴, -(alkylene)ₘ-SO₂—NR³R⁴, -(alkylene)ₘ-N(R³)—SO₂—R⁵, -(alkylene)ₘ-N(R³)—SO₂—NR³R⁴, -(alkylene)ₘ-N(R³)—C(O)—OR⁵, -(alkylene)ₘ-N(R³)—C(S)—OR⁵ or -(alkylene)ₘ-N(R³)—SO₂—R⁵; wherein:

the alkyl, haloalkyl, alkenyl, alkynyl, cycloalkyl, cycloalkenyl, heterocyclyl, aryl, heteroaryl, arylalkyl, heteroarylalkyl, cycloalkylalkyl and heterocycloalkyl may be further independently substituted with one or more of the following groups:
-(alkylene)ₘ-CN, -(alkylene)ₘ-OR⁵*, -(alkylene)ₘ-S(O)ₙ—R⁵*, -(alkylene)ₘ-NR³*R⁴*, -(alkylene)ₘ-C(O)—R⁵* -(alkylene)ₘ-C(═S)R⁵*, -(alkylene)ₘ-C(═O)OR⁵*, -(alkylene)ₘ-OC(═O)R⁵*, -(alkylene)ₘ-C(S)—OR⁵*, -(alkylene)ₘ-C(O)—NR³*R⁴*, -(alkylene)ₘ-C(S)—NR³*R⁴*, -(alkylene)ₘ-N(R³*)—C(O)—NR³*R⁴*, -(alkylene)ₘ-N(R³*)—C(S)—NR³*R⁴*, -(alkylene)ₘ-N(R³*)—C(O)—R⁵* -(alkylene)ₘ-N(R³*)—C(S)—R⁵* -(alkylene)ₘ-O—C(O)—NR³*R⁴*, -(alkylene)ₘ-O—C(S)—NR³*R⁴*, -(alkylene)ₘ-SO₂—NR³*R⁴*, -(alkylene)ₘ-N(R³*)—SO₂—R⁵*, -(alkylene)ₘ-N(R³*)—SO₂—NR³*R⁴*, -(alkylene)ₘ-N(R³*)—C(O)—OR⁵* -(alkylene)ₘ-N(R³*)—C(S)—OR⁵* or -(alkylene)ₘ-N(R³*)—SO₂—R⁵*
n is 0, 1 or 2, and m is 0 or 1:

R³* and R⁴* at each occurrence are independently:

(i) hydrogen or

(ii) alkyl, alkenyl, alkynyl, cycloalkyl, heterocyclyl, aryl, heteroaryl, cycloalkylalkyl, heterocycloalkyl, arylalkyl or heteroarylalkyl, any of which may be optionally independently substituted with one or more R^{x} groups as allowed by valance; or R³* and R⁴* together with the nitrogen atom to which they are attached may combine to form a heterocyclic ring optionally independently substituted with one or more R^{x} groups as allowed by valance; and
R⁶ is H or lower alkyl, -(alkylene)ₘ-heterocyclyl, -(alkylene)ₘ-heteroaryl, -(alkylene)ₘ-NR³R⁴, -(alkylene)ₘ-C(O)—NR³R⁴; -(alkylene)ₘ-O—R⁵, -(alkylene)ₘ-S(O)ₙ—R⁵ or -(alkylene)ₘ-S(O)ₙ—NR³R⁴, any of which may be optionally independently substituted with one or more R^{x} groups as allowed by valance, and wherein two R groups bound to the same or adjacent atoms may optionally combine to form a ring;
or a pharmaceutically acceptable salt, prodrug, isotopic variant (e.g., completely or partially deuterated form) or solvate thereof.

In some embodiments, the pharmaceutically acceptable salt of the compound as shown in Formula I is hydrochloride.

In some embodiments, the compound has a structure of Formula I and R⁶ is absent.

In some embodiments, the R^{x} in the Formula I is not further substituted.

In some embodiments, the R⁸ in the Formula I is hydrogen or C₁-C₃ alkyl.

In some embodiments, the R¹¹ in the Formula I is hydrogen or C₁-C₃ alkyl.

In some embodiments, the compound comprises a compound as shown in Formula Ia:

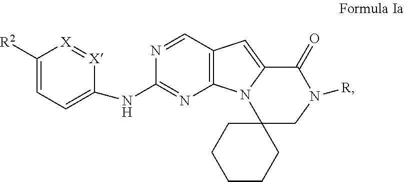

In some embodiments, the X in the Formula I or Formula Ia is C.

In some embodiments, the X′ in the Formula I or Formula Ia is N.

In some embodiments, the R in the Formula I or Formula Ia is hydrogen or C₁-C₃ alkyl.

In some embodiments, the R² in the Formula I or Formula Ia is -(alkylene)ₘ-heterocyclyl, -(alkylene)ₘ-NR³R⁴, -(alkylene)ₘ-C(O)—NR³R⁴, -(alkylene)ₘ-C(O)—O-alkyl or -(alkylene)ₘ-OR⁵, any of which may be optionally independently substituted with one or more R^{x} groups as allowed by valance, and wherein two RY groups bound to the same or adjacent atoms may optionally combine to form a ring, and wherein mis 0 or 1.

In some embodiments, the R² in the Formula I or Formula Ia is -(alkylene)ₘ-heterocyclyl, -(alkylene)ₘ˜NR³R⁴, -(alkylene)ₘ-C(O)—NR³R⁴-(alkylene)ₘ-C(O)—O-alkyl or -(alkylene)ₘ-OR⁵, which is not further substituted.

In some embodiments, m in the R² in the Formula I or Formula Ia is 1.

In some embodiments, alkylene in the R² in the Formula I or Formula Ia is methylene.

In some embodiments, the R² in the Formula I or Formula Ia is

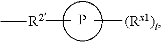

wherein, R²* is a bond, alkylene, -(alkylene)ₘ-O-(alkylene)ₘ-, -(alkylene)ₘ-C(O)-(alkylene)ₘ-, -(alkylene)ₘ-S(O)₂-(alkylene)ₘ- and ˜(alkylene)ₘ-NH-(alkylene)ₘ-, wherein each m is independently 0 or 1;
P is a 4- to 8-membered mono- or bicyclic saturated heterocyclyl group;
each R^{x1} is independently -(alkylene)ₘ-(C(O))ₘ-(alkylene)ₘ-(N(R^{N}))ₘ-(alkyl)ₘ, wherein each m is independently 0 or 1 provided at least one m is 1; —(C(O)—O-alkyl; -(alkylene)ₘ-cycloalkyl wherein m is 0 or 1; —N(R^{N})-cycloalkyl; —C(O)-cycloalkyl; -(alkylene)ₘ-heterocyclyl wherein m is 0 or 1; or —N(R^{N})-heterocyclyl; —C(O)-heterocyclyl; —S(O))₂-(alkylene)ₘ wherein m is 1 or 2, wherein:
R^{N} is H, C₁ to C₄ alkyl or C₁ to C₆ heteroalkyl, and
wherein two R^{x1} can, together with the atoms to which they attach on P, which may be the same atom, form a ring; and
t is 0, 1 or 2.

In some embodiments, the each R^{x1} in the R² is optionally substituted by unsubstituted alkyl, halogen or hydroxyl.

In some embodiments, R^{x1} in the R² is hydrogen or unsubstituted C₁-C₄alkyl.

In some embodiments, the at least one R^{x1} in the R² is -(alkylene)ₘ-heterocyclyl, wherein m is 0 or 1.

In some embodiments, the R² in the Formula I or Formula Ia is

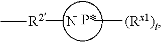

wherein P* is a 4- to 8-membered mono- or bicyclic saturated heterocyclyl group.

In some embodiments, the R² in the Formula I or Formula Ia is

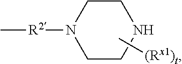

In some embodiments, the R² in the Formula I or Formula Ja is

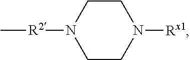

In some embodiments, the R² in the Formula I or Formula Ia is

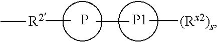

wherein, R²* is a bond, alkylene, -(alkylene)ₘ-O-(alkylene)ₘ-, -(alkylene)ₘ-C(O)-(alkylene)ₘ-, -(alkylene)ₘ-S(O)₂-(alkylene)ₘ- and -(alkylene)ₘ-NH-(alkylene)ₘ, wherein each m is independently 0 or 1;
P is a 4- to 8-membered mono- or bicyclic saturated heterocyclyl group;
P1 is a 4- to 6-membered monocyclic saturated heterocyclyl group; and
each R^{X2} is independently hydrogen or alkyl; and s is 0, 1 or 2.

In some embodiments, the R² in the Formula I or Formula

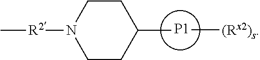

In some embodiments, P1 in the R² comprises at least one nitrogen.

In some embodiments, any alkylene in R^{2*} of any one embodiment above is not further substituted:

In some embodiments, the R^{2*} in the R² is a bond.

In some embodiments, the R^{x1} in the R² is hydrogen, methyl or propyl.

In some embodiments, the R²

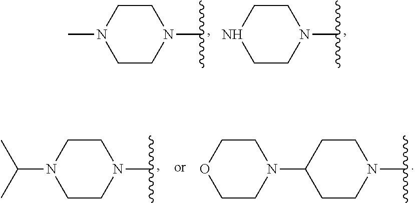

In some embodiments, the compound is selected from the following group.

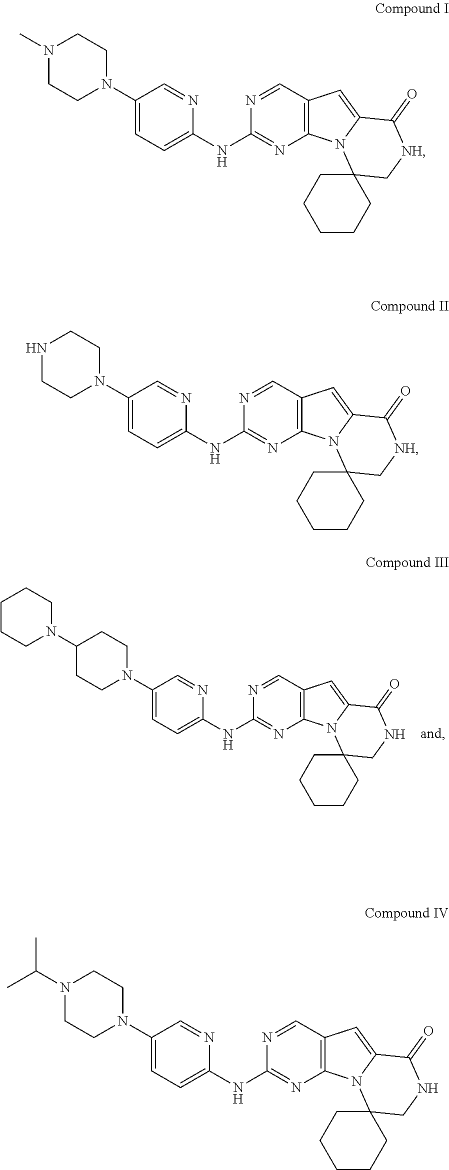

In some embodiments, the compound is

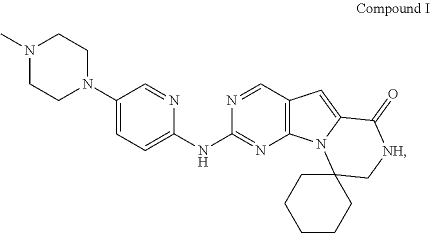 [SMILES: CN1CCN(c2ccc(Nc3ncc4cc5n(c4n3)C3(CCCCC3)CNC5=O)nc2)CC1]

and its hydrochloride form.

In some embodiments, the compound is

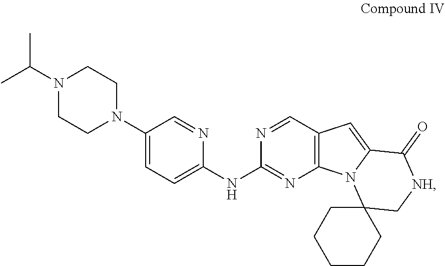 [SMILES: CC(C)N1CCN(c2ccc(Nc3ncc4cc5n(c4n3)C3(CCCCC3)CNC5=O)nc2)CC1]

and its hydrochloride form

In some embodiments, the chemotherapy comprises administration of a chemotherapeutic agent.

In some embodiments, the chemotherapeutic agent is a cytotoxic agent.

In some embodiments, the chemotherapeutic agent is selected from one or more of the following group: a DNA synthesis inhibitor, an RNA synthesis inhibitor, a protein synthesis inhibitor, a cell division inhibitor, a DNA base analogue, a topoisomerase inhibitor and/or a telomerase synthesis inhibitor.

In some embodiments, the chemotherapy is administered in combination with one or more other therapies.

In some embodiments, the chemotherapeutic agent is selected from fluorouracil, oxaliplatin, irinotecan, topotecan, etoposide, cisplatin, docetaxel, gemcitabine, carboplatin, methotrexate, doxorubicin, cytarabine, vinorelbine and capecitabine, and a combination thereof.

In some embodiments, the chemotherapeutic agent is selected from 5-fluorouracil, irinotecan and oxaliplatin, and a combination thereof.

In some embodiments, the chemotherapy-associated gastrointestinal side effects comprise gastrointestinal side effects caused by the chemotherapeutic agent.

In some embodiments, the chemotherapy-associated gastrointestinal side effects comprise gastrointestinal adverse events that occur or deteriorate after the administration of the chemotherapeutic agent.

In some embodiments, the gastrointestinal adverse events will occur or deteriorate about 1 hr, about 2 hrs, about 3 hrs, about 4 hrs, about 5 hrs, about 6 hrs. about 7 hrs, about 8 hrs, about 9 hrs, about 10 hrs, about 11 hrs, about 12 hrs, about 1 day, about 2 days, about 4 days, about 7 days, about 2 weeks, about 3 weeks, about 1 month, about 2 months or longer after the administration of the chemotherapeutic agent, in the absence of prevention or treatment.

In some embodiments, the gastrointestinal side effects comprise gastric mucosal injury diseases and/or intestinal mucosal injury diseases.

In some embodiments, the gastrointestinal side effects comprise diarrhea, abdominal pain, nausea, vomiting, mucositis, loss of appetite, gastric ulcer, gastritis, constipation, enteritis, intestinal perforation, intestinal bleeding, ulcer and/or intestinal necrosis.

In some embodiments, the gastrointestinal side effects comprise diarrhea, constipation and/or enteritis.

In some embodiments, the severity of the gastrointestinal side effects is Grade 1 or above, Grade 2 or above, Grade 3 or above, Grade 4 or above, or Grade 5, as evaluated in accordance with NCI-CTCAE.

In some embodiments, the subject comprises a cancer patient.

In some embodiments, the medicament does not substantially affect the therapeutic effect of the chemotherapeutic agent.

In some embodiments, the medicament is administered about 0.5 hrs, about 1 hr, about 2 hrs, about 3 hrs, about 4 hrs, about 5 hrs, about 6 hrs, about 7 hrs, about 8 hrs, about 9 hrs, about 10 hrs, about 11 hrs, about 12 hrs, about 13 hrs, about 14 hrs, about 15 hrs, about 16 hrs, about 17 hrs, about 18 hrs, about 19 hrs, about 20 hrs or longer before the administration of the chemotherapy.

In some embodiments, the medicament is administered about 0.5˜12 hrs before the administration of the chemotherapy.

In some embodiments, the medicament is prepared for oral administration.

In some embodiments, the medicament is prepared for injection administration.

In some embodiments, the medicament is prepared for administration through subcutaneous injection, intramuscular injection, intraperitoneal injection and/or intravenous injection.

In some embodiments, the medicament further comprises one or more other active ingredients.

In another aspect, the present application provides a method of preventing, relieving and/or treating chemotherapy-associated gastrointestinal side effects, comprising administering to a subject in need thereof the compound as shown in Formula I or a pharmaceutically acceptable salt thereof.

In another aspect, the present application provides the compound as shown in Formula I or a pharmaceutically acceptable salt thereof, which is used for preventing, relieving and/or treating chemotherapy-associated gastrointestinal side effects in a subject.

In another aspect, the present application provides a pharmaceutical combination, comprising the compound as shown in Formula I or a pharmaceutically acceptable salt thereof and a chemotherapeutic agent.

In another aspect, the present application provides a kit, comprising the compound as shown in Formula I or a pharmaceutically acceptable salt thereof and a chemotherapeutic agent.

Other aspects and advantages of the present application will be readily apparent to those skilled in the art from the following detailed description. Only the exemplary embodiments of the present application are shown and described in the following detailed description. As will be appreciated by those skilled in the art, the disclosure of the present application allows persons skilled in the art to modify the disclosed embodiments without departing from the spirit and scope of the invention involved in the present application. Accordingly, the drawings and the description in the specification of the present application are merely exemplary, and not restrictive.

### BRIEF DESCRIPTION OF THE DRAWINGS

Specific features of the invention involved in the present application are set forth in the appended claims. The features and advantages of the invention involved in the present application can be better understood by referring to the exemplary embodiments detailed hereinafter and the accompanying drawings. A brief description of the drawings is as follows:

FIG. 1 shows the diarrhea photographs of typical mice in the chemotherapeutic agent groups of examples 1-201.

FIG. 2 shows partial results of diarrhea grading in the control group, the chemotherapeutic agent group, and the group of the compound as shown in Formula I of examples 1-201.

FIG. 3 shows the photographs showing the number and shape of faeces in the control group, the chemotherapeutic agent group, and the group of the compound as shown in Formula I of examples 202-364.

FIG. 4 shows partial results of feces reduction rate in the chemotherapeutic agent group, and the group of the compound as shown in Formula I of examples 202-364.

FIG. 5 shows partial results of diarrhea grading in the control group, the chemotherapeutic agent group, the group of the compound as shown in Formula I of examples 365-384.

FIG. 6 shows partial results of feces reduction rate in the control group, and the group of the compound as shown in Formula I of examples 385-405.

### DETAILED DESCRIPTION

The embodiments of the present invention are described below by way of specific examples, and persons skilled in the art can readily appreciate other advantages and effects of the present invention from the disclosure of the present specification.

### Term Definition

In the present application, the term “alkyl”, either alone or within other terms such as “haloalkyl” and “alkylamino”, generally refers to linear or branched radicals having one to about twelve carbon atoms. “Lower alkyl” radicals generally have one to about six carbon atoms. Examples of such radicals include methyl, ethyl, n-propyl, isopropyl, n-butyl, isobutyl, sec-butyl, tert-butyl, pentyl, isopentyl, hexyl, and the like. The term “alkylene” embraces bridging divalent linear or branched alkyl radicals. Examples may include methylene, ethylene, propylene, isopropylene and the like.

In the present application, the term “alkenyl” generally refers to linear or branched radicals having at least one carbon-carbon double bond and two to about twelve carbon atoms. “Lower alkenyl” radicals have two to about six carbon atoms. Examples of alkenyl radicals include vinyl, propenyl, allyl, propenyl, butenyl and 4-methylbutenyl. The terms “alkenyl” and “lower alkenyl” embrace radicals having “cis” and “trans” orientations or alternatively, “E” and “Z” orientations.

In the present application, the term “alkynyl” generally refers to linear or branched radicals having at least one carbon-carbon triple bond and having two to about twelve carbon atoms. “Lower alkynyl” radicals have two to about six carbon atoms. Examples of such radicals include propargyl, butynyl, and the like. Alkyl, alkenyl and alkynyl radicals may be optionally substituted with one or more functional groups such as halo, hydroxyl, nitro, amino, cyano, haloalkyl, aryl, heteroaryl, heterocyclyl and the like.

In the present application, the term “alkylamino” generally embraces “N-alkylamino” and “N,N-dialkylamino”, where amino groups are independently substituted with one alkyl radical and two alkyl radicals, respectively. “Lower alkylamino” radicals generally have one or two alkyl radicals of one to six carbon atoms attached to a nitrogen atom. Suitable alkylamino radicals may be monoalkylamino or dialkylamino, such as N-methylamino, N-ethylamino, N,N-dimethylamino or N,N-diethylamino.

In the present application, the term “halo” generally refers to halogen, such as fluorine, chlorine, bromine or iodine atoms.

In the present application, the term “haloalkyl” generally refers to radicals wherein any one or more of the alkyl carbon atoms is substituted with one or more halo. Examples may include monohaloalkyl, dihaloalkyl and plyhaloalkyl radicals including perhaloalkyl. For example, a monohaloalkyl radical may have an iodo, bromo, chloro or fluoro atom within the radical. Dihaloalkyl and polyhaloalkyl radicals may have two or more of the same halo atoms or a combination of different halo radicals. “Lower haloalkyl” may embrace radicals having 1-6 carbon atoms. Examples of haloalkyl radicals may include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Perfluoroalkyl” generally refers to an alkyl radical having all hydrogen atoms replaced with fluoro atoms. Examples may include trifluoromethyl and pentafluoroethyl.

In the present application, the term “aryl”, alone or in combination with other terms, generally refers to a carbocyclic aromatic system containing one or two rings, wherein such rings may be attached together in a fused manner. The term “aryl” generally refers to aromatic radicals, such as phenyl, naphthyl, indenyl, tetrahydronaphthyl and indanyl. More preferred aryl is phenyl. Said “aryl” group may have 1 or more substituents, such as lower alkyl, hydroxyl, halo, haloalkyl, nitro, amino, alkoxy, lower alkylamino and the like. An aryl group may be optionally substituted with one or more functional groups such as halo, hydroxyl, nitro, amino, cyano, haloalkyl, aryl, heteroaryl, heterocyclyl and the like.

In the present application, the term “heterocyclyl” (or “heterocyclic ring”) generally refers to saturated and partially saturated heteroatom-containing ring radicals, where the heteroatoms may be selected from nitrogen, sulfur and oxygen. Heterocyclic rings comprise monocyclc 6-8-membered rings and 5-16-membered bicyclic ring systems (which can include bridged fused and spiro-fused bicyclic ring systems). It does not include rings containing —O—O—, —O—S— or —S—S— portions. Said “heterocyclyl” radical may have 1 to 3 substituents such as hydroxyl, Boc, halo, haloalkyl, amino, lower alkyl, lower aralkyl, oxo, lower alkoxy, amino, lower alkylamino and the like.

Examples of saturated heterocyclyl radicals include saturated 3- to 6-membered heteromonocyclic groups containing 1 to 4 nitrogen atoms (e.g., pyrrolidinyl, imidazolidinyl, piperidinyl, pyrrolinyl, piperazinyl); saturated 3- to 6-membered heteromonocyclic groups containing 1 to 2 oxygen atoms and 1 to 3 nitrogen atoms (e.g., morpholinyl); saturated 3 to 6-membered heteromonocyclic groups containing 1 to 2 sulfur atoms and 1 to 3 nitrogen atoms (e.g., thiazolidinyl). Examples of partially saturated heterocyclyl radicals include dihydrothienyl, dihydropyranyl, dihydrofuryl, dihydrothiazolyl, and the like.

Particular Examples of partially saturated and saturated heterocyclyl radicals may include pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-λ′benzo[d]isothiazol-6-yl, dihydropyranyl, dihydrofuryl and dihydrothiazolyl and the like.

Heterocyclo groups also include radicals where heterocyclic radicals are fused/condensed with aryl radicals: condensed heterocyclic groups containing 1 to 5 nitrogen atoms, e.g., indolyl, isoindolyl, indolizinyl, benzimidazolyl, quinolyl, isoquinolyl, indazolyl, benzotrizaolyl, tetrahydropyridazinyl (e.g., tetrazolo[1,5-b]pyridazinyl); unsaturated condensed heterocyclic groups containing 1 to 2 oxygen atoms and 1 to 3 nitrogen atoms (e.g., benzoxazolyl, benzoxadiazolyl); unsaturated condensed heterocyclic groups containing 1 to 2 sulfur atoms and 1 to 3 nitrogen atoms (e.g., benzothiazolyl, benzothiadiazolyl); and saturated, partially unsaturated and unsaturated condensed heterocyclic groups containing 1 to 2 oxygen or sulfur atoms (e.g., benzofuryl, benzothienyl, 2,3-dihydrobenzo[1,4]dioxanyl and dihydrobenzofuryl).

In the present application, the term “heteroaryl” generally refers to aryl ring systems containing one or more heteroatoms selected from the group consisting of O, N and S, wherein the ring nitrogen and sulfur atom(s) are optionally oxidized, and nitrogen atom(s) are optionally quarternized. Examples include unsaturated 5- to 6-membered heteromonocyclic groups containing 1 to 4 nitrogen atoms, e.g., pyrrolyl, imidazolyl, pyrazolyl, 2-pyridyl, 3-pyridyl, 4-pyridyl, pyrimidyl, pyrazinyl, pyridazinyl, triazolyl (e.g., 4H-1,2,4-triazolyl, 1H-1,2,3-triazolyl, 2H-1,2,3-triazolyl); unsaturated 5- to 6-membered heteromonocyclic groups containing an oxygen atom, e.g., pyranyl, 2-furyl, 3-furyl, etc.; unsaturated 5- to 6-membered heteromonocyclic groups containing a sulfur atom, e.g., 2-thienyl, 3-thienyl, etc.; unsaturated 5- to 6-membered heteromonocyclic groups containing 1 to 2 oxygen atoms and 1 to 3 nitrogen atoms, e.g., oxazolyl, isoxazolyl, oxadiazolyl (e.g., 1,2,4-oxadiazolyl, 1,3,4-oxadiazolyl, 1,2,5-oxadiazolyl); unsaturated 5- to 6-membered heteromonocyclic groups containing 1 to 2 sulfur atoms and 1 to 3 nitrogen atoms, e.g., thiazolyl, thiadiazolyl (e.g., 1,2,4-thiadiazolyl, 1,3,4-thiadiazolyl, 1,2,5-thiazdiaolyl).

In the present application, the term “heteroarylalkyl” generally refers to alkyl radicals substituted with a heteroaryl group. Examples include pyridylmethyl and thienylethyl.

In the present application, the term “sulfonyl”, whether used alone or together with other terms such as alkylsulfonyl, denotes divalent radicals —SO₂— respectively.

In the present application, the term “carboxy” or “carboxyl”, whether used alone or together with other terms such as “carboxyalkyl”, denotes —C(O)—OH.

In the present application, the term “carbonyl”, whether used alone or together with other terms such as “aminocarbonyl”, denotes —C(O)—.

In the present application, the term “aminocarbonyl” generally refers to an amido group of Formula —C(O)—NH₂.

In the present application, the term “heterocycloalkyl” generally refers to heterocyclic-substituted alkyl radicals. Examples include piperidinylmethyl and morpholinylethyl.

In the present application, the term “arylalkyl” generally refers to aryl-substituted alkyl radicals. Examples include benzyl, diphenylmethyl and phenylethyl. The aryl in said arylalkyl may be additionally substituted with halo, alkyl, alkoxy, haloalkyl and haloalkoxy.

In the present application, the term “cycloalkyl” generally refers to saturated carbocyclic groups of 3 to 10 carbons. Lower cycloalkyl groups include C₃-C₆ rings. Examples include cyclopentyl, cyclopropyl and cyclohexyl. Cycloalkyl groups may be optionally substituted with one or more functional groups such as halo, hydroxyl, nitro, amino, cyano, haloalkyl, aryl, heteroaryl, heterocyclyl and the like.

In the present application, the term “cycloalkylalkyl” generally refers to cycloalkyl-substituted alkyl radicals. “Lower cycloalkylalkyl” radicals are cycloalkyl radicals attached to alkyl radicals having one to six carbon atoms. Examples include cyclohexylmethyl. The cycloalkyl in said radicals may be additionally substituted with halo, alkyl, alkoxy and hydroxyl.

In the present application, the term “cycloalkenyl” includes carbocyclic groups having one or more carbon-carbon double bonds, including “cycloalkyldienyl” compounds. Examples include cyclopentenyl, cyclopentadienyl, cyclohexenyl and cycloheptadienyl.

In the present application, the term “oxo” generally refers to an oxygen atom attached with a double bond.

In the present application, the term “nitro” generally refers to —NO₂.

In the present application, the term “cyano” generally refers to —CN.

In the present application, the term “prodrug” generally refers to a compound which when administered to a host in vivo is converted into the parent drug. As used herein, the term “parent drug” generally refers to any of the presently described chemical compounds that are useful to treat any of the disorders described herein, or to control or improve the underlying cause or symptoms associated with any physiological or pathological disorder described herein in a host (e.g., a human). Prodrugs can be used to achieve any desired effect, including to enhance properties of the parent drug or to improve the pharmaceutic or pharmacokinetic properties of the parent drug. Prodrug strategies exist which provide choices in modulating the conditions for in vivo generation of the parent drug, all of which are deemed included herein. Nonlimiting examples of prodrug strategies include the following covalent attachments: removable groups or removable portions of groups, for example, but not limited to acylation, phosphorylation, phosphonylation, phosphoramidate derivatives, amidation, reduction, oxidation, esterification, alkylation, other carboxy derivatives, sulfoxy or sulfone derivatives, carbonylation or anhydride, among others.

In the present application, the term “chemotherapy” generally refers to a therapy that uses a chemotherapeutic agent to treat tumors, which can lead to the death of cancer cells, or interfere with the division, repair, growth and/or function of cancer cells. The chemotherapeutic agent comprises a chemical or biological substance which can cause the death of cancer cells or interfere with the growth, division, repair and/or function of cancer cells. For example, the chemotherapeutic agent may comprise cytotoxic agens, cell inhibitors and antitumor agents which can kill tumor cells, inhibit the growth or metastasis of tumor cells or disrupt the cell cycle of rapidly proliferating cells. The chemotherapeutic agent comprises natural compounds found in animals and plants, or artificial chemical substances. In the present application, the chemotherapeutic agents have low selectivity on tumor cells and normal cells, so they may also interfere with the growth, division, repair and/or function of normal cells (e.g., bone marrow cells, hair follicle cells or digestive tract cells), finally resulting in the death of normal cells. The chemotherapeutic agents in the present application may not include tumor-targeted medicaments, i.e., the chemotherapeutic agents in the present application do not include substances which can specifically recognize tumor cells, tumor tissues, tumor organs or proteins specifically expressed by tumor cells (e.g., tumor-associated antigens or tumor specific antigens). Because of genetic mutation or other factors, some kinases or phosphoric acid kinases are activated, causing the reproduction of cancer cells. The chemotherapeutic agents of the present application do not include those kinase inhibitors which prevent the division of cancer cells by inhibiting abnormally activated kinases, either. The chemotherapeutic agents of the present application do not include antibodies and/or angiogenesis inhibitors, either.

In the present application, examples of chemotherapy may include, but not limited to, alkylating agents, e.g., mechlorethamine, ethyleneimine compounds, alkyl sulfonates and other compounds having alkylating effects, e.g. nitrosourea, cisplatin and dacarbazine; antimetabolites, e.g., folic acid, purine or pyrimidine antagonists; mitotic inhibitors, e.g. vinca alkaloid and podophyllotoxin derivatives; cytotoxic antibiotics and camptothecin derivatives. The chemotherapy may also include amifostine cisplatin, dacarbazine (DTIC), dactinomycin, streptomycin, cyclophosphamide, carmustine (BCNU), lomustine (CCNU), doxorubicin, doxorubicin liposome, gemcitabine, erythromycin, daunorubicin liposome procarbazine, mitomycin, cytarabine, etoposide, methotrexate, 5-fluorouracil (5-FU), vinblastine, vincristine, bleomycin, paclitaxel, Docetaxel, Aldesleukin, Asparaginase, busulfan, carboplatin, cladribine, camptothecin, irinotecan, 10-hydroxyl-7-ethyl-camptothecin (SN38), floxuridine, fludarabine, hydroxyurea, ifosfamide, idarubicin, Mesna, interferon α, interferon β, mitoxantrone, topotecan, Lupron, megestrol, Melphalan, mercaptopurine, plicamycin, mitotane, Oncaspar, pegaspargase, pentostatin, pipobroman, plicamycin, streptozocin, tamoxifen, teniposide, testosterone, thioguanine, thiotepa, uramustine, vinorelbine, chlorambucil aromatase inhibitor and a combination thereof.

In the present application, the term “cytotoxic agent” generally refers to a reagent for inhibiting the biological process of cells or reducing the viability or proliferative potential of cells. The cytotoxic agent may act in various ways, for example, but not limited to, through inducing DNA damages, inducing cell cycle arrest, inhibiting DNA synthesis, inhibiting transcription, inhibiting translation or protein synthesis, inhibiting cell division or inducing apoptosis.

In the present application, the term “continuous administration” generally refers to repeated administration of the same medicament every day. Repeated administration every day may be once a day, twice a day, 3 or more times a day. For example, continuous administration may be administration once a day for at least more than 2 days. For example, a continuous administration time of 2 days may mean administration once a day (twice, 3 or more times) continuously for 2 days. For example, a continuous administration time of 3 days may mean administration once a day (twice, 3 or more times) continuously for 3 days. For example, a continuous administration time of 5 days may mean administration once a day (twice, 3 or more times) continuously for 5 days. For example, a continuous administration time of 7 days may mean administration once a day (twice, 3 or more times) continuously for 7 days. For example, a continuous administration time of 10 days may mean administration once a day (twice, 3 or more times) continuously for 10 days. For example, a continuous administration time of 14 days may mean administration once a day (twice, 3 or more times) continuously for 14 days. For example, a continuous administration time of more than 7 days may mean administration once a day (twice, 3 or more times) continuously for more than 7 days.

In the present application, the term “noncontinuous administration” generally refers to an administration mode in which the same medicament of the same type administered repeatedly is not administered every day in an administration cycle, also known as intermittent administration and/or pulsed administration. Noncontinuous administration may be regular or irregular. In noncontinuous administration, in an administration cycle, for any continuous period of time, if the administration is performed every day and continuously, it also belongs to the case of continuous administration in this period of time.

In the present application, the term “does not substantially affect” generally refers to that as compared to the therapeutic effect of using the chemotherapy alone, the use of the combination of the medicament and the chemotherapy of the present application has a comparable therapeutic effect, or does not produce a significant disadvantage. For example, for any subject, as compared to the therapeutic effect of using the chemotherapy alone, the use of the combination of the medicament and the chemotherapy causes the same degree of tumor size reduction, or the reduction degree is not less than about 5%, not less than about 4%, not less than about 3%, not less than about 2%, not less than about 1%, not less than about 0.5%, not less than about 0.1%, not less than about 0.01%, not less than about 0.001% or smaller.

In the present application, the term “gastrointestinal adverse events” generally refers to harmful and undesirable reactions, effects, actions, consequences, results or influences associated with the gastrointestinal tract or displayed in the gastrointestinal tract area, which are caused by a certain medicament or other medical treatments such as chemotherapy or surgery. They are also known as gastrointestinal adverse effects, gastrointestinal adverse influences or gastrointestinal adverse consequences. The gastrointestinal adverse events comprise adverse events in various parts (e.g., digestive tracts and glands) of the digestive system. For example, gastrointestinal adverse events may include, but not limited to, abdominal distension, abdominal pain, anal fissure, anal fistula, anal bleeding, anal mucositis, anal necrosis, anal pain, anal stenosis, anal ulcer, ascites, hiccup, cecal bleeding, cheilitis, chylous ascites, colitis, colonic fistula, colonorrhagia, colonic obstruction, colonic perforation, colonic stricture, colonic ulcer, constipation, dental caries, diarrhea, xerostomia, duodenal fistula, duodenal bleeding, duodenal obstruction, duodenuml perforation, duodenal stenosis, duodenal ulcer, dyspepsia, dysphagia, enterocolitis, intestinal fistula, esophageal fistula, esophageal bleeding, esophageal necrosis, esophageal obstruction, esophageal pain, esophageal perforation, esophageal stenosis, esophageal ulcer, esophageal variceal bleeding, oesophagitis, fecal incontinence, flatus, gastric fistula, gastrorrhagia, gastric necrosis, gastric perforation, gastric stenosis, gastric ulcer, gastritis, gastroesophageal reflux disease, gastrointestinal diseases, gastrointestinal fistula, gastrointestinal pain, gastroparesis, gum pain, hemorrhoidal bleeding, haemorrhoids, ileal fistula, ileal bleeding, ileocleisis, ileal perforation, ileal stenosis, ileal ulcer, ileus, intraperitoneal hemorrhage, jejunal fistula, jejunal bleeding, jejunal obstruction, jejunal perforation, jejunal stenosis, jejunal ulcer, chilalgia, lower gastrointestinal hemorrhage, malabsorption, oral mucositis, nausea, gastric obstruction, oral fistula, oral dysaesthesia, oral hemorrhage, oral pain, pancreatic-duct stenosis, pancreatic fistula, pancreatic bleeding, pancreatic necrosis, pancreatitis, periodontal diseases, peritoneal necrosis, rectitis, rectal rupture, rectofistula, rectal bleeding, rectal mucositis, rectal necrosis, rectal obstruction, rectal pain, rectal perforation, rectostenosis, rectal ulcer, retroperitoneal hemorrhage, salivary duct inflammation, salivary gland fistula, small bowel mucositis, small bowel obstruction, small bowel perforation, small bowel stenosis, small bowel ulcer, diarrhea, dental developmental disturbance, dental stain, toothache, appendicitis, upper gastrointestinal hemorrhage, visceral artery ischemia, vomiting.

In the present application, the term “gastrointestinal side effects” generally refers to harmful and adverse effects associated with the gastrointestinal tract or displayed in the gastrointestinal tract area, which are caused by a preventive medicine or a therapeutic medicine. Adverse effects are always undesirable, but undesirable effects are not necessarily adverse. The adverse effects of the preventive medicine or the therapeutic medicine may be harmful, unsuitable or dangerous. Gastrointestinal side effects include side effects in various parts (e.g., digestive tract and gland) of the digestive system. Chemotherapy-associated gastrointestinal side effects may refer to any abnormal clinical manifestations of gastrointestinal tract associated with the time of using the chemotherapeutic agent, but there may be no causal relationship between these abnormal manifestations and the administration of the chemotherapeutic agent.

In the present application, the term “gastric mucosal injury diseases” generally refers to the diseases or disorders with symptoms of gastric mucosal injury, which can include abnormal color, bleeding spots, congestion and erosion of the gastric mucosa.

In the present application, the term “intestinal mucosal injury diseases” generally refers to the diseases or disorders with symptoms of intestinal mucosal injury, which can include abnormal color, bleeding spots, congestion and erosion of the intestinal mucosa.

In the present application, the term “diarrhea” generally refers to a disease or disorder with the features of increased peristalsis frequency and/or relaxation or watery peristalsis. In the present application, the diarrhea may occur or deteriorate after administration of the chemotherapy. In some cases, the description of the standardized definition of diarrhea can refer to various versions of Common Terminology Criteria for Adverse Events (CTCAE) issued by National Cancer Institute (NCI). In some cases, diarrhea can also be evaluated referring to animal experiments: for example, the severity of diarrhea is scored in accordance with the method of Akinobu Kurita (Cancer Chemother Pharmacol 2000; 46:211-20.), wherein, Grade 0: normal feces; Grade 1: mild diarrhea, the stools is slightly wet and soft; Grade 2: moderate diarrhea, loose stools and mild perianal contamination; Grade 3: severe diarrhea, watery stools and accompanied by severe perianal staining.

In the present application, the term “constipation” generally refers to a disease or disorder with the features of irregular defecation, infrequent defecation or difficult defecation of intestines. In the present application, the constipation may occur or deteriorate after administration of the chemotherapy. The description of the standardized definition of constipation can refer to various versions of Common Terminology Criteria for Adverse Events (CTCAE) issued by National Cancer Institute (NCI).

In the present application, the term “enteritis” generally refers to inflammation of the intestines, including colitis, enteritis, enterocolitis, duodenitis, ileitis, appendicitis, rectitis and/or jeiunitis. In the present application, the enteritis may occur or deteriorate after administration of the chemotherapy. The description of the standardized definition of enteritis can refer to various versions of Common Terminology Criteria for Adverse Events (CTCAE) issued by National Cancer Institute (NCI).

In the present application, the term “in the absence of prevention or treatment” generally refers to a condition without performing prevention or treatment on gastrointestinal adverse events, and without taking measures to make the gastrointestinal adverse events to be weakened or disappear. The measures to make the gastrointestinal adverse events to be weakened or disappear may be, for example, administration of a medicament or other means to prevent or treat gastrointestinal adverse events, stopping the administration of medicaments or other means that cause gastrointestinal adverse events, including administration of the compound as shown in Formula I or the medicament of the present application. In the present application, after administering the chemotherapeutic agent of the present application, if no measures are taken to make the gastrointestinal adverse events to be weakened or disappear, the gastrointestinal adverse events will occur or deteriorate 1 hr, 2 hrs, 3 hrs, 4 hrs, 5 hrs, 6 hrs, 7 hrs, 8 hrs, 9 hrs, 10 hrs, 11 hrs, 12 hrs, 1 day, 2 days, 4 days, 7 days, 2 weeks, 3 weeks, 1 month, 2 months or longer after the administration of the chemotherapy. If the compound as shown in Formula I or the medicament of the present application is administered, the gastrointestinal adverse events may be weakened or disappear.

In the present application, the term “NCI-CTCAE” generally refers to a standardized definition of adverse events issued by National Cancer Institute (NCI)-Common Terminology Criteria for Adverse Events (CTCAE), which is used to describe the severity of organ toxicity in patients treated for cancer. With the development of scientific evidence, the criteria can be updated continuously. In the present application, “NCI-CTCAE” may comprise any version of “NCI-CTCAE”. In CTCAE V5.0, constipation and diarrhea are defined into 5 grades respectively, as shown in Table 1 and Table 2.

TABLE 1
Standardized definition on the grading
of constipation in CTCAE V5.0

| CTCAE | |
|---|---|
| Grading | Criteria |
| Grade 1 | Incidental or intermittent symptoms; occasionally |
| | employing stool softener, laxatives, |
| | antisecosis or enteroclysis |
| Grade 2 | persistent symptoms in need of long-term use |
| | of laxatives or enteroclysis; limited |
| | instrumental activities of daily living (ADL) |
| Grade 3 | Constipation with manual evacuation indications; |
| | limited self-care |
| Grade 4 | Life-threatening consequences; indicating |
| | emergency interventions |
| Grade 5 | Death |

TABLE 2
Standardized definition on the grading of diarrhea in CTCAE V5.0

| CTCAE | |
|---|---|
| Grading | Criteria |
| Grade 1 | An increasement of <4 times of defecation |
| | compared with the basic standard every day; |
| | compared with the baseline, the output of |
| | neostomy increases slightly |
| Grade 2 | An increasement of 4-6 times of defecation |
| | compared with the basic standard every day; |
| | compared with the baseline, the output of neostomy |
| | increases moderately; limited instrumental ADL |
| Grade 3 | An increasement of >=7 times of defecation |
| | compared with the basic standard every |
| | day; indicating hospitalization, compared with |
| | the baseline, the output of neostomy |
| | increases significantly; limited self-care |
| Grade 4 | Life-threatening consequences; indicating |
| | emergency interventions |
| Grade 5 | Death |

In the present application, the term “cancer” generally refers to any medical conditions mediated by the growth, proliferation or metastasis of tumors or malignant cells and causing solid tumors and nonsolid tumors (e.g., leukemia). The cancer in the present application may include, but not limited to, malignant epithelial tumors (epithelium-derived cancers), lung cancer (e.g., non-small cell lung cancer), breast cancer, skin cancer, bladder cancer, colon cancer, gastrointestinal (GI) cancer, prostatic cancer, pancreatic cancer, uterine cancer, cervical cancer, ovarian cancer, esophagus cancer, head and neck cancer, gastric cancer and throat cancer.

### DETAILED DESCRIPTION OF THE INVENTION

Compound as shown in Formula I

In one aspect, the present application provides a use of a compound as shown in Formula I in preparing a medicament, which can be used for preventing, relieving and/or treating chemotherapy-associated gastrointestinal side effects in a subject:

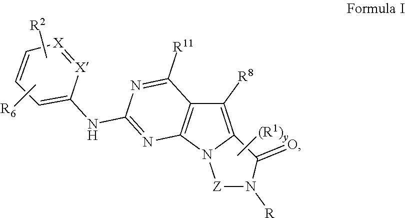

wherein,
Z is —(CH)_{X}—, wherein X is 1, 2, 3 or 4, or —O—(CH)_{Z}—, wherein z is 2, 3 or 4;
X and X′ are each independently CH or N;
R, R⁸ and R¹¹ are each independently H, C₁-C₃ alkyl or haloalkyl, cycloalkyl or cycloalkyl containing one or more heteroatoms selected from N, O or S, -(alkylene)ₘ-C₃-C₈ cycloalkyl, -(alkylene)ₘ-aryl, -(alkylene)ₘ-heterocyclyl, -(alkylene)ₘ-heteroaryl, -(alkylene)ₘ-NR³R⁴, -(alkylene)ₘ-C(O)—NR³R⁴, -(alkylene)ₘ-O—R⁵, -(alkylene)ₘ-S(O)ₙ—R⁵, or -(alkylene)ₘ-S(O)ₙ—NR³R⁴, any of which may be optionally independently substituted with one or more R groups as allowed by valance, and wherein two R^{x} groups bound to the same or adjacent atoms may optionally combine to form a ring, and wherein m is 0 or 1, n is 0, 1 or 2;
R¹ is independently aryl, alkyl, cycloalkyl or haloalkyl, wherein each of the alkyl, cycloalkyl and haloalkyl optionally comprises O or N heteroatoms in place of a carbon in the chain, and two R¹'s on adjacent ring atoms or on the same ring atom together with the ring atom(s) to which they are attached optionally form a 3-8-membered cycle;
y is 0, 1, 2, 3 or 4;
R² is -(alkylene)ₘ-heterocyclyl, -(alkylene)ₘ-heteroaryl, -(alkylene)ₘ-NR³R⁴, -(alkylene)ₘ-C(O)—NR³R⁴, -(alkylene)ₘ-C(O)—O-alkyl; -(alkylene)ₘ-O—R⁵, -(alkylene)ₘ-S(O)ₙ—R⁵ or -(alkylene)ₘ-S(O)ₙ—NR³R⁴, any of which may be optionally independently substituted with one or more R^{x} groups as allowed by valance, and wherein two R groups bound to the same or adjacent atoms may optionally combine to form a ring, and wherein m is 0 or 1, n is 0, 1 or 2;
R³ and R⁴ at each occurrence are independently:
(i) hydrogen or
(ii) alkyl, cycloalkyl, heterocyclyl, aryl, heteroaryl, cycloalkylalkyl, heterocycloalkyl, arylalkyl or heteroarylalkyl, any of which may be optionally independently substituted with one or more R^{x} groups as allowed by valance, and wherein two R^{x} groups bound to the same or adjacent atoms may optionally combine to form a ring; or R³ and R⁴ together with the nitrogen atom to which they are attached may combine to form a heterocyclic ring optionally independently substituted with one or more R^{x} groups as allowed by valance, and wherein two R^{x} groups bound to the same or adjacent atoms may optionally combine to form a ring;
R⁵ and R⁵* at each occurrence are:
(i) hydrogen or
(ii) alkyl, alkenyl, alkynyl, cycloalkyl, heterocyclyl, aryl, heteroaryl, cycloalkylalkyl, heterocycloalkyl, arylalkyl or heteroarylalkyl, any of which may be optionally independently substituted with one or more R^{x} groups as allowed by valance;
R^{x} at each occurrence is independently halo, cyano, nitro, oxo, alkyl, haloalkyl, alkenyl, alkynyl, cycloalkyl, cycloalkenyl, heterocyclyl, aryl, heteroaryl, arylalkyl, heteroarylalkyl, cycloalkylalkyl, heterocycloalkyl, -(alkylene)ₘ-OR⁵, -(alkylene)ₘ-O-alkylene-OR⁵, -(alkylene)ₘ-S(O)ₙ—R⁵, -(alkylene)ₘ-NR³R⁴, -(alkylene)ₘ-CN, -(alkylene)ₘ-C(O)—R⁵, -(alkylene)ₘ-C(S)—R⁵, -(alkylene)ₘ-C(O)—OR⁵, -(alkylene)ₘ-O—C(O)—R⁵, -(alkylene)ₘ-C(S)—OR⁵, -(alkylene)ₘ-C(O)-(alkylene)ₘ-NR³R⁴, -(alkylene)ₘ-C(S)—NR³R⁴, -(alkylene)ₘ-N(R³)—C(O)—NR³R⁴, -(alkylene)ₘ-N(R³)—C(S)—NR³R⁴, -(alkylene)ₘ-N(R³)—C(O)—R⁵, -(alkylene)ₘ-N(R³)—C(S)—R⁵, -(alkylene)ₘ-O—C(O)—NR³R⁴, -(alkylene)ₘ-O—C(S)—NR³R⁴, -(alkylene)ₘ-SO₂—NR³R⁴, -(alkylene)ₘ-N(R³)—SO₂—R⁵, -(alkylene)ₘ-N(R³)—SO₂—NR³R⁴, -(alkylene)ₘ-N(R³)—C(O)—OR⁵, -(alkylene)ₘ-N(R³)—C(S)—OR⁵ or -(alkylene)ₘ-N(R³)—SO₂—R⁵; wherein:
the alkyl, haloalkyl, alkenyl, alkynyl, cycloalkyl, cycloalkenyl, heterocyclyl, aryl, heteroaryl, arylalkyl, heteroarylalkyl, cycloalkylalkyl and heterocycloalkyl may be further independently substituted with one or more of the following groups:
-(alkylene)ₘ-CN, -(alkylene)ₘ-OR⁵*, -(alkylene)ₘ-S(O)ₙ—R⁵*, -(alkylene)ₘ-NR³*R⁴*, -(alkylene)ₘ-C(O)—R⁵*, -(alkylene)ₘ-C(═S)R⁵* -(alkylene)ₘ-C(═O)OR⁵* -(alkylene)ₘ-OC(═O)R⁵* -(alkylene)ₘ-C(S)—OR⁵*, -(alkylene)ₘ-C(O)—NR³*R⁴*, -(alkylene)ₘ-C(S)—NR³*R⁴*, -(alkylene)ₘ-N(R³*)—C(O)—NR³*R⁴*, -(alkylene)ₘ-N(R³*)—C(S)—NR³*R⁴*, -(alkylene)ₘ-N(R³*)—C(O)—R⁵*, -(alkylene)ₘ-N(R³*)—C(S)—R⁵*, -(alkylene)ₘ-O—C(O)—NR³*R⁴*, -(alkylene)ₘ-O—C(S)—NR³*R⁴*, -(alkylene)ₘ-SO₂—NR³*R⁴*, -(alkylene)ₘ-N(R³*)—SO₂—R⁵*, -(alkylene)ₘ-N(R³*)—SO₂—NR³*R⁴*, -(alkylene)ₘ-N(R³*)—C(O)—OR⁵*, -(alkylene)ₘ-N(R³*)—C(S)—OR⁵* or -(alkylene)ₘ-N(R³*)—SO₂—R⁵*,
n is 0, 1 or 2, and m is 0 or 1;
R³* and R⁴* at each occurrence are independently:
(i) hydrogen or
(ii) alkyl, alkenyl, alkynyl, cycloalkyl, heterocyclyl, aryl, heteroaryl, cycloalkylalkyl, heterocycloalkyl, arylalkyl or heteroarylalkyl, any of which may be optionally independently substituted with one or more R^{x} groups as allowed by valance; or R³* and R⁴* together with the nitrogen atom to which they are attached may combine to form a heterocyclic ring optionally independently substituted with one or more RY groups as allowed by valance; and
R⁶ is H or lower alkyl, -(alkylene)ₘ-heterocyclyl, -(alkylene)ₘ-heteroaryl, -(alkylene)ₘ-NR³R⁴, -(alkylene)ₘ-C(O)—NR³R⁴; -(alkylene)ₘ-O—R⁵, -(alkylene)ₘ-S(O)ₙ—R⁵ or -(alkylene)ₘ-S(O)ₙ—NR³R⁴, any of which may be optionally independently substituted with one or more R^{x} groups as allowed by valance, and wherein two R^{x} groups bound to the same or adjacent atoms may optionally combine to form a ring:

or a pharmaceutically acceptable salt, prodrug, isotopic variant (e.g., completely or partially deuterated form) or solvate thereof.

In another aspect, the present application provides a method of preventing, relieving and/or treating chemotherapy-associated gastrointestinal side effects in a subject, comprising administering the compound as shown in Formula I.

In the present application, the term “a compound as shown in Formula I” may be known as “a compound of Formula I”, “Formula I”. Such a term is also defined as comprising all the forms of the compound of Formula I, including hydrate, solvate, isomer, crystalline and non-crystalline form, isomorphism, polymorphism, and metabolite. For example, the compound of Formula I or a pharmaceutically acceptable salt thereof may be present in an unsolvated or solvated form. When the binding force of solvent or water thereto is relatively strong, the coordination compound has a definite stoichiometry that is not affected by humidity. However, if the binding force of solvent or water thereto is relatively weak, e. g., in a channel solvate and a hygroscopic compound, the content of water/solvent will depend on the humidity and drying conditions. In this case, non-stoichiometry is normal.

The “compound of Formula I” may comprise chiral carbon atom(s). In the present application, the carbon-carbon bonds in the compound of Formula I may be represented by solid lines, solid wedges, or dot wedges. Depicting the bond to a chiral carbon atom with solid lines means that all the potential stereoisomers (e. g., specific enantiomers, racemates, etc.) on the carbon atom are comprised. The compound of the present application may comprise more than one chiral carbon atoms. In these compounds, depicting the bond to a chiral carbon atom with solid lines is intended to indicate that all the potential stereoisomers should be encompassed. For example, unless otherwise indicated, the compound of Formula I may present as enantiomers, diastereomers, or racemates and mixtures thereof. Depicting the bond to one or more chiral carbon atoms in the compound of Formula I with solid lines and depicting the bond to another chiral carbon atom in the same compound with solid or dot wedges indicate the presence of a mixture of diastereomers.

The compound of the present application may be present as inclusion compounds or other coordination compounds. The present application encompasses complexes, e.g., inclusion compounds, drug-host inclusion complex, wherein in contrast to the aforesaid solvates, drug and host are present in stoichiometric or non-stoichiometric amount. The present application further comprises a coordination compound of Formula I comprising two or more organic and/or inorganic components that may be stoichiometric or non-stoichiometric. The resultant complex may be ionized, partially ionized, or unionized.

The stereoisomer of Formula I comprises cis- and trans-isomers, optical isomers, e. g., R and S enantiomers, diastereomers, geometrical isomers, rotamers, conformational isomers and tautomers, the compound of Formula I, including the compounds exhibiting one or more of the aforesaid types of isomerisms, and mixtures thereof (e.g., racemate and diastereomer pair). The stereoisomer further comprises acid or base addition salts in which the counter ions have optical activity, e.g., D-lactate or L-lysine, or racemates, e.g., DL-tartrate or DL-arginine.

When any racemate crystallizes, there may be two different types of crystals. The first type is the racemic compounds (true racemates) as described above, wherein a homogeneous form of crystal is produced and comprises two enantiomers in equimolar amounts. The second type is a racemic mixture or agglomerate, wherein two forms of crystals are produced in equimolar amounts, each comprising a single enantiomer.

The compound of Formula I can exhibit tautomerism and structural isomerism. For example, the compound of Formula I may be present in several tautomeric forms, comprising enol and imine forms, and ketone and enamine forms; as well as geometric isomers and their mixtures. All these tautomeric forms are encompassed within the scope of the compound of Formula I. Tautomers are present in solution as a mixture of tautomers. In the solid form, one tautomer generally dominates. Even if one tautomer may be described, the present invention also comprises all the tautomers of the compound of Formula I.

The present application may further comprise isotope-labeled compounds that are the same as that of Formula I except that one or more atoms thereof are replaced with atoms having atomic mass or mass number different from those found in nature. Isotopes which may be incorporated into the compound of Formula I comprise isotopes of hydrogen, carbon, nitrogen, oxygen, phosphorus, fluorine and chlorine, e. g., but are not limited to: ²H, ³H, ¹³C, ¹⁴C, ¹⁵N, ¹⁸O, ¹⁷O, ³¹P, ³²P, ³⁵S, ¹⁸F, and ³⁶Cl. Some isotope-labeled compounds of Formula I. e. g., into which radioisotopes (e. g., ³H and ¹⁴C) are incorporated, may be used for measuring the distribution of drugs and/or substrate tissues due to their easy preparation and detectability. Heavier isotopes, such as, ²H, can provide certain therapeutic advantages due to its greater metabolic stability, e. g., longer half-life in the body or lower dose requirements. The isotope-labeled compounds of Formula I generally may be prepared with isotope-labeled reagents in stead of non-isotope-labeled reagents.

The compound of the present application may be used as salts derived from inorganic or organic acids. Some compounds have advantages, such as, enhanced drug stability at different temperatures and humidities, or desired solubilities in water/oil due to the physical properties of one or more salts. In some cases, the salts of the compound can also be adjuvants for use in the separation, purification, and/or resolution of the compound.

In the present application, the compound has a structure of Formula I and R⁶ may be absent.

In the present application, the R^{x} in the Formula I may be not further substituted.

In the present application, the R⁸ in the Formula I may be hydrogen or C₁-C₃ alkyl.

In the present application, the R¹¹ in the Formula I may be hydrogen or C₁-C₃ alkyl.

In the present application, the compound may comprise a compound as shown in Formula Ia:

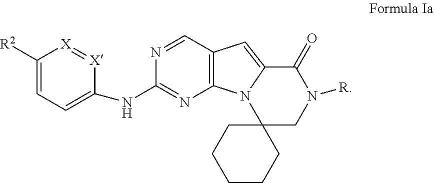

In the present application, the X in the Formula I or Formula Ia may be C.

In the present application, the X′ in the Formula I or Formula Ia may be N.

In the present application, the R in the Formula I or Formula Ia may be hydrogen or C₁-C₃ alkyl.

In the present application, the R² in the Formula I or Formula Ia may be -(alkylene)ₘ-heterocyclyl, -(alkylene)ₘ-NR³R⁴, -(alkylene)ₘ-C(O)—NR³R⁴, -(alkylene)ₘ-C(O)—O-alkyl or -(alkylene)ₘ-OR⁵, any of which may be optionally independently substituted with one or more R^{x} groups as allowed by valance, and wherein two R^{x} groups bound to the same or adjacent atoms may optionally combine to form a ring, and wherein m is 0 or 1.

In the present application, the R² in the Formula I or Formula Ia may be -(alkylene)ₘ-heterocyclyl, -(alkylene)ₘ-NR³R⁴, -(alkylene)ₘ-C(O)—NR³R⁴, -(alkylene)ₘ-C(O)—O-alkyl or -(alkylene)ₘ-OR⁵, which is not further substituted.

In the present application, m in the R² in the Formula I or Formula Ia may be 1.

In the present application, the alkylene in the R² in the Formula I or Formula Ia may be methylene.

In the present application, the R² in the Formula I or Formula Ta may be

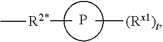

wherein, R^{2*} may be a bond, alkylene, -(alkylene)ₘ-O-(alkylene)ₘ-, -(alkylene)ₘ-C(O)-(alkylene)ₘ-, -(alkylene)ₘ-S(O)₂-(alkylene)ₘ- and -(alkylene)ₘ-NH-(alkylene)ₘ2, wherein each m may be independently 0 or 1:
P may be a 4- to 8-membered mono- or bicyclic saturated heterocyclyl group;
each R^{x1} may be independently -(alkylene)ₘ-(C(O))ₘ-(alkylene)ₘ-(N(R^{N})ₘ-(alkyl)ₘ, wherein each m may be independently 0 or 1 provided at least one m may be 1; —(C(O))—O-alkyl; -(alkylene)-cycloalkyl, wherein m may be 0 or 1; —N(R^{N})-cycloalkyl; —C(O)-cycloalkyl; -(alkylene)ₘ-heterocyclyl, wherein m may be 0 or 1; or —N(R^{N})-heterocyclyl; —C(O)-heterocyclyl; —S(O)-(alkylene)ₘ, wherein m may be 1 or 2, wherein:
R^{N} may be H, C₁ to C₄ alkyl or C₁ to C₆ heteroalkyl, and
wherein two R^{x1} can, together with the atoms to which they attach on P, which may be the same atom, form a ring; and
t is 0, 1 or 2.

In the present application, the each R^{x1} in the R² may be optionally substituted by unsubstituted alkyl, halogen or hydroxyl.

In the present application, R^{x1} in the R² may be hydrogen or unsubstituted C₁-C₄ alkyl.

In the present application, the at least one R^{x1} in the R² may be -(alkylene)ₘ-heterocyclyl, wherein m is 0 or 1.

In the present application, the R² in the Formula I or Formula Ta is

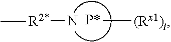

wherein P* may be a 4- to 8-membered mono- or bicyclic saturated heterocyclyl group.

In the present application, the R² in the Formula I or Formula Ia may be

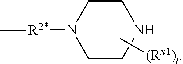

In the present application, the R² in the Formula I or Formula Ia may be

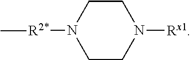

In the present application, the R² in the Formula I or Formula Ia may be

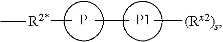

wherein, R^{2*} s a bond, alkylene, -(alkylene)ₘ-O-(alkylene)ₘ-, -(alkylene)ₘ-C(O)-(alkylene)ₘ-(alkylene)ₘ-S(O)₂-(alkylene)ₘ- and -(alkylene)ₘ-NH-(alkylene)ₘ-, wherein each m is independently 0 or 1, s is 0, 1 or 2;
P is a 4- to 8-membered mono- or bicyclic saturated heterocyclyl group;
P1 is a 4- to 6-membered monocyclic saturated heterocyclyl group; and
each R^{X2} may be independently hydrogen or alkyl; and s is 0, 1 or 2.

In the present application, the R² in the Formula I or Formula Ia may be

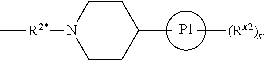

In the present application, P1 in the R² may comprise at least one nitrogen.

In the present application, any alkylene in R^{2*} in any one of the above embodiments may be not further substituted.

In the present application, the R^{2*} in the R² may be a bond.

In the present application, the R^{x1} in the R² may be hydrogen, methyl or propyl:

For example, the R² may be

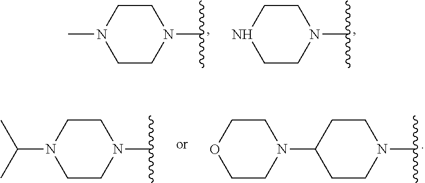

For example, the compound as shown in Formula I may be selected from the following group:

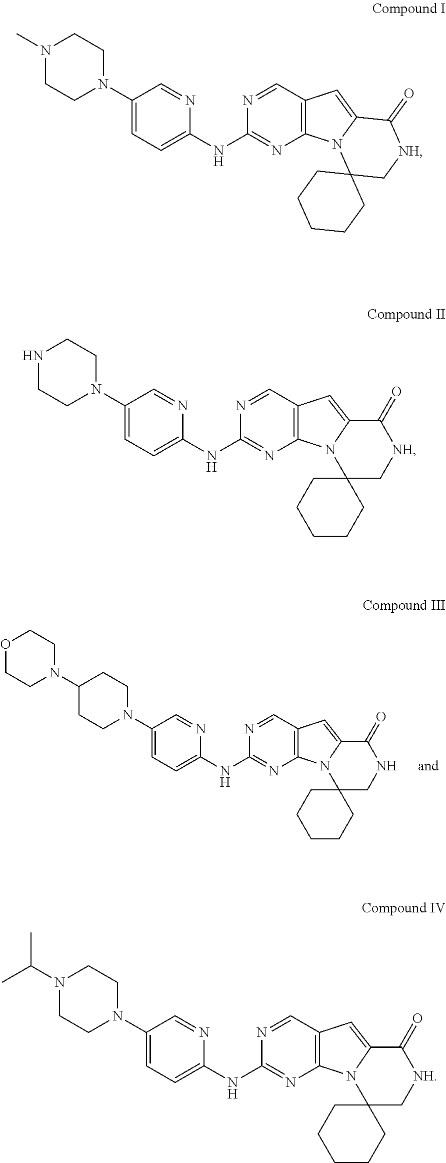 [SMILES: CN1CCN(c2ccc(Nc3ncc4cc5n(c4n3)C3(CCCCC3)CNC5=O)nc2)CC1 ; O=C1NCC2(CCCCC2)n2c1cc1cnc(Nc3ccc(N4CCNCC4)cn3)nc12 ; O=C1NCC2(CCCCC2)n2c1cc1cnc(Nc3ccc(N4CCC(N5CCOCC5)CC4)cn3)nc12 ; "and" ; CC(C)N1CCN(c2ccc(Nc3ncc4cc5n(c4n3)C3(CCCCC3)CNC5=O)nc2)CC1]

For example, the compound as shown in Formula I may be

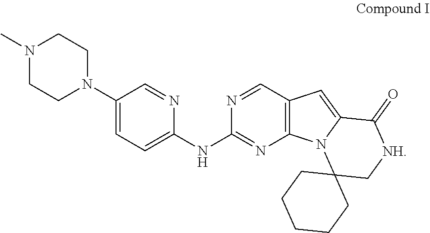 [SMILES: CN1CCN(c2ccc(Nc3ncc4cc5n(c4n3)C3(CCCCC3)CNC5=O)nc2)CC1]

For example, the compound as shown in Formula I may be the hydrochloride form of

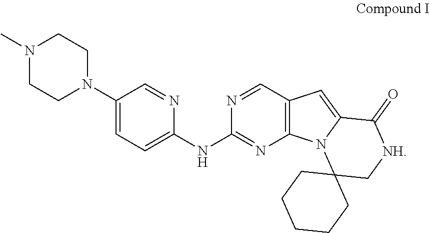 [SMILES: CN1CCN(c2ccc(Nc3ncc4cc5n(c4n3)C3(CCCCC3)CNC5=O)nc2)CC1]

For example, the compound shown in Formula I may be

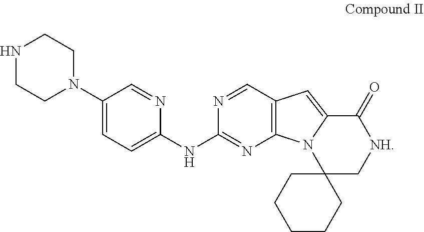 [SMILES: O=C1NCC2(CCCCC2)n2c1cc1cnc(Nc3ccc(N4CCNCC4)cn3)nc12]

For example, the compound as shown in Formula I may be the hydrochloride form of

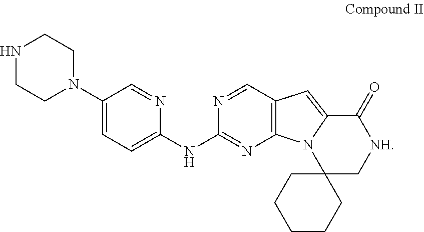 [SMILES: O=C1NCC2(CCCCC2)n2c1cc1cnc(Nc3ccc(N4CCNCC4)cn3)nc12]

For example, the compound shown in Formula I may be

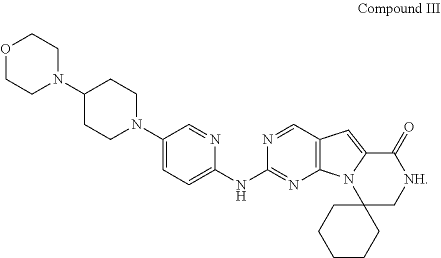 [SMILES: O=C1NCC2(CCCCC2)n2c1cc1cnc(Nc3ccc(N4CCC(N5CCOCC5)CC4)cn3)nc12]

For example, the compound as shown in Formula I may be the hydrochloride form of

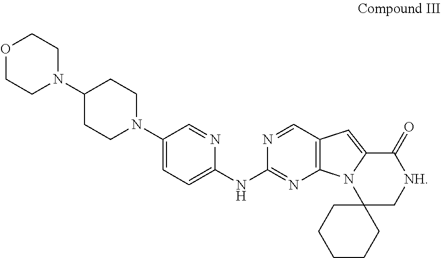 [SMILES: O=C1NCC2(CCCCC2)n2c1cc1cnc(Nc3ccc(N4CCC(N5CCOCC5)CC4)cn3)nc12]

For example, die compound shown in Formula I may be

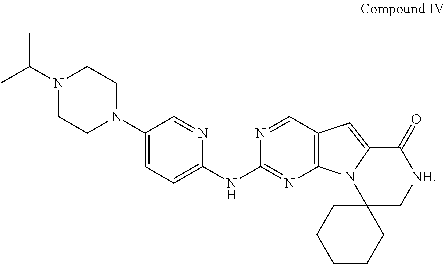 [SMILES: CC(C)N1CCN(c2ccc(Nc3ncc4cc5n(c4n3)C3(CCCCC3)CNC5=O)nc2)CC1]

For example, the compound as shown in Formula I may be the hydrochloride form of

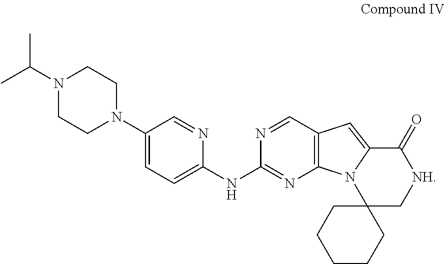 [SMILES: CC(C)N1CCN(c2ccc(Nc3ncc4cc5n(c4n3)C3(CCCCC3)CNC5=O)nc2)CC1]

### Chemotherapy

In the present application, the chemotherapeutic agent used in the chemotherapy may be any one kind of compound or medicament used for preventing, relieving and/or treating cancers selected from those known to persons skilled in the art. In terms of the mechanism of action, the chemotherapy may comprise a DNA synthesis inhibitor, an RNA synthesis inhibitor, a protein synthesis inhibitor, a cell division inhibitor, a DNA base analogue, a topoisomerase inhibitor and/or a telomerase synthesis inhibitor.

In the present application, the chemotherapeutic agent may be toxic to immunologic effector cells. In the present application, the chemotherapeutic agent may inhibit cell growth. In the present application, the administered chemotherapeutic agent may be a chemotherapeutic agent for DNA damages. In the present application, the chemotherapeutic agent is a protein synthesis inhibitor, a DNA-damage chemotherapeutic agent, an alkylating agent, a topoisomerase inhibitor, an RNA synthesis inhibitor, a DNA composite binding agent, a thiolate alkylating agent, a guanine alkylating agent, a tubulin binding agent, a DNA polymerase inhibitor, an anti-cancer enzyme, a RACI inhibitor, a thymidylic acid synthetase inhibitor, an oxazophosphorine compound, an integrin inhibitor, such as cilengitide, camptothecin or homocamptothecin, an antifolate, a folic acid antimetabolite, a telomerase inhibitor and/or a telomere DNA-binding compound.

For example, the alkylating agent may comprise alkyl sulfonates, such as busulfan, improsulfan and piposulfan; aziridines, such as benzodizepa, carboquone, meturedepa and uredepa; ethylene imines and methyl melamines, such as altretamine, triethylene melamine, triethylene phosphamide, triethylene thiophosphamide and trimethylol melamine; mechlorethamines, such as chlorambucil, chlornaphazine, cyclophosphamide, estramustine, dichloromethyl diethylamine, mechlorethamine oxide hydrochloride, Melphalan, novembichine, phenesterine, prednimustine, trofosfamide and uracil mustard; and nitrosoureas, such as carmustine, chlorozotocin, fotemustine, lomustine, nimustine and ranimustine. Other chemotherapeutic agents may comprise daunorubicin, doxorubicin, idarubicin, epirubicin, mitomycin and streptozotocin. Antimetabolites for the chemotherapy may comprise gemcitabine, mercaptopurine, thioguanine, cladribine, fludarabine phosphate, fluorouracil (5-FU), floxuridine, cytarabine, pentostatin, methotrexate, azathioprine, acyclovir, adenine β-1-D-arabinoside, amethopterin, aminopterin, 2-aminopurine, aphidicolin, 8-azaguanine, azaserine, 6-azauracil, 2′-azido-2′-deoxynucleoside, 5-bromodeoxycytidine, cytosine β-1-D-arabinoside, diazoxonorleucine, dideoxynucleoside, 5-fluorodeoxy cytidine, 5-fluorodeoxyuridine and hydroxyurea.

For example, the protein synthesis inhibitor may comprise abrin, aurin tricarboxylic acid, chloramphenicol, colicin E3, cycloheximide, diphtheria toxin, edeine A, emetine, erythromycin, ethionine, fluoride, 5-fluorotryptophan, fusidic acid, guanylyl methylene bisphosphonate and guanylyl imino diphosphate, kanamycin, kasugamycin, kirromycin and O-methylthreonine. Other protein synthesis inhibitors comprise modeccin, neomycin, norvaline, pactamycin, paromomycine, puromycin, ricin, Shiga toxin, showdomycin, sparsomycin, spectinomycin, streptomycin, tetracycline, thiostreptone and trimethoprim.

For example, the DNA synthesis inhibitor may include an alkylating agent, such as dimethyl sulfate, mechlorethamine and sulfur mustard; an intercalating agent, such as acridine dyes, actinomycin, anthracenes, benzopyrene, ethidium bromide, propidium diiodide-intertwining; a topoisomerase inhibitor, such as irinotecan, teniposide, coumermycin, nalidixic acid, novobiocin and oxolinic acid; a cell division inhibitor, including demecolcine, mitoxantrone, colchicine, vinblastine and vincristine; and other medicaments, such as distamycin and netropsin.

In the present application, the chemotherapeutic agent may be a DNA composite binding agent, e.g., camptothecin or etoposide; a thiolate alkylating agent, e.g. nitrosourea, BCNU, CCNU, ACNU or fotesmustine; a guanine alkylating agent, e.g., temozolomide; a tubulin binding agent, such as vinblastine, vincristine, vinorelbine, vinflunine, cryptophycin 52, halichondrin such as halichondrin B, aplysiatoxin such as aplysiatoxin 10 and aplysiatoxin 15, hemiasterlins (such as hemiasterlin A and hemiasterlin B), colchicine, combrestatins, 2-methoxy estradiol, E7010, paclitaxel, Docetaxel, epothilone, discodermolide; a DNA polymerase inhibitor, e.g., cytarabine; anti-cancer enzyme, e.g. asparaginase; a RACI inhibitor, e.g., 6-thioguanine; a thymidylic acid synthetase inhibitor, e.g., capecitabine or 5-FU; an oxazophosphorine compound, e.g., Endoxan; an integrin inhibitor, e.g., cilengitide; an antifolate, e.g., pralatrexate; a folic acid antimetabolite, e.g., pemetrexed; or camptothecin or homocamptothecin, e.g., diflomotecan.

In the present application, the compound as shown in Formula I may be used for preventing, relieving and/or treating the above one or more chemotherapy-associated gastrointestinal side effects.

In the present application, the compound as shown in Formula I may be used for preventing, relieving and/or treating gastrointestinal side effects associated with the administration of the following chemotherapeutic agents: fluorouracil, oxaliplatin, irinotecan, topotecan, etoposide, cisplatin, docetaxel, gemcitabine, carboplatin, methotrexate, doxorubicin, cytarabine, vinorelbine and capecitabine, and a combination thereof.

In the present application, the compound as shown in Formula I may be used for preventing, relieving and/or treating gastrointestinal side effects associated with the administration of the following chemotherapeutic agents: fluorouracil, oxaliplatin, irinotecan (CPT-11), tetrahydrofolic acid, and a combination thereof. In the present application compound as shown in Formula I may be used for preventing, relieving and/or treating gastrointestinal side effects associated with the following chemotherapies: 5-fluorouracil, irinotecan and oxaliplatin, and a combination thereof.

In the present application, the chemotherapeutic agents may not include tumor-targeted medicaments. In the present application, the chemotherapeutic agents may not include those kinase inhibitors which prevent the division of cancer cells by inhibiting abnormally activated kinase. In the present application, the chemotherapeutic agents may not include an antibody and/or an angiogenesis inhibitor. For example, the chemotherapeutic agent may not include a tyrosine kinase inhibitor.

In the present application, the chemotherapeutic agent may be administered continuously. For example, the chemotherapy may be administered continuously for 2 days, 3 days, 4 days, 5 days, 6 days, 7 days, 8 days, 9 days or more days. In some embodiments, the days for the continuous administration of the chemotherapy are no more than 7 days, for example, the chemotherapy may be administered continuously for 2 days, 3 days, 4 days, 5 days, 6 days or 7 days. In the present application, the administration frequency of the chemotherapy may be once a day, twice a day, 3 times a day or others.

In the present application, the chemotherapeutic agent may be administered noncontinuously. In the present application, the administration frequency of the chemotherapy may be once every two days, once every three days, twice every three days or others.

For example, the continuous administration time of the chemotherapy may be no more than 7 days. For example, the continuous administration time of the chemotherapy may be less than 7 days (e.g., administration continuously for 2 days, 3 days, 4 days, 5 days or 6 days).

For example, the administration cycle of the chemotherapy may be 3 days, 5 days, 7 days, 2 weeks, 20 days, 1 month, 2 months or longer. The administration mode, administration route and/or administration time of the chemotherapeutic agent may refer to the package insert of the chemotherapeutic agent.

In the present application, one or more different chemotherapies may be used. In some embodiments, the compound as shown in Formula I may be used in combination with one or more other cancer therapies. The other cancer therapies may be common methods used for treating cancers in the art, e.g., cytotoxic anticancer agent, immunotherapeutic anticancer agent or hormonotherapeutic anticancer agent. In accordance with the present application, the medicament used for treating a cancer may also be used in combination with radiotherapy or surgery. In some embodiments, in the case of the combination use of the compound as shown in Formula I and other anticancer agents, they can be administered to the subject at the same time, or separately administered at a certain interval.

### Gastrointestinal Side Effects

In the present application, the compound as shown in Formula I can prevent, relieve and/or treat gastrointestinal side effects associated with the administration of chemotherapy in a subject. In the present application, chemotherapy-associated gastrointestinal side effects may mean that the gastrointestinal side effects are caused by the administration of the chemotherapy and will occur or deteriorate after the administration of the chemotherapy. In the absence of prevention or treatment, the gastrointestinal side effects will occur or deteriorate about 1 hr, about 2 hrs, about 3 hrs, about 4 hrs, about 5 hrs, about 6 hrs, about 7 hrs, about 8 hrs, about 9 hrs, about 10 hrs, about 11 hrs, about 12 hrs, about 1 day, about 2 days, about 4 days, about 7 days, about 2 weeks, about 3 weeks, about 1 month, about 2 months or longer after the administration of the chemotherapy.

In some embodiments, before administering the chemotherapy to the subject, the subject does not develop the gastrointestinal side effects; after administering the chemotherapy to the subject, the subject develops the gastrointestinal side effects.

In some embodiments, before administering the chemotherapy to the subject, the subject has developed the gastrointestinal side effects; after administering the chemotherapy to the subject, the severity of the gastrointestinal side effects in the subject increases. In the present application, after the administration of the chemotherapy, the symptoms of gastrointestinal side effects (e.g., diarrhea, enteritis or constipation) in the subject may deteriorate by at least about 10%, e.g., by about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or higher. For example, in accordance with the criteria of NCI-CTCAE, after administering the chemotherapy to the subject, the severity of the gastrointestinal side effects (e.g., diarrhea, enteritis or constipation) in the subject increases from Grade 1 to Grade 2, from Grade 1 to Grade 3, from Grade 1 to Grade 4, from Grade 1 to Grade 5, from Grade 2 to Grade 3, from Grade 2 to Grade 4, from Grade 2 to Grade 5, from Grade 3 to Grade 4, from Grade 3 to Grade 5 or from Grade 4 to Grade 5. For example, in accordance with the method of Akinobu Kurita, after administering the chemotherapy to the subject, the scoring of constipation in the subject increases from Grade 0 to Grade 1, from Grade 0 to Grade 2, from Grade 0 to Grade 3, from Grade 1 to Grade 2, from Grade 1 to Grade 3 or from Grade 2 to Grade 3.

In some embodiments, the gastrointestinal side effects comprise gastric mucosal injury diseases and/or intestinal mucosal injury diseases.

In some embodiments, the gastrointestinal side effects comprise diarrhea, abdominal pain, nausea, vomiting, mucositis, loss of appetite, gastric ulcer, gastritis, constipation, enteritis, intestinal perforation, intestinal bleeding, ulcer and/or intestinal necrosis. In some embodiments, the gastrointestinal side effects comprise abnormal defecation. In some embodiments, the gastrointestinal side effects comprise diarrhea and/or constipation.

In some embodiments, the chemotherapy-associated gastrointestinal side effects comprise chemotherapy-associated gastric mucosal injury diseases and/or chemotherapy-associated intestinal mucosal injury diseases.

In some embodiments, the chemotherapy-associated gastrointestinal side effects comprise chemotherapy-associated diarrhea, chemotherapy-associated abdominal pain, chemotherapy-associated nausea, chemotherapy-associated vomiting, chemotherapy-associated mucositis, chemotherapy-associated loss of appetite, chemotherapy-associated gastric ulcer, chemotherapy-associated gastritis, chemotherapy-associated constipation, chemotherapy-associated enteritis, chemotherapy-associated intestinal perforation, chemotherapy-associated intestinal bleeding, chemotherapy-associated ulcer and/or chemotherapy-associated intestinal necrosis. In some embodiments, the chemotherapy-associated gastrointestinal side effects comprise chemotherapy-associated abnormal defecation. In some embodiments, the chemotherapy-associated gastrointestinal side effects comprise chemotherapy-associated diarrhea and/or chemotherapy-associated constipation.

In the present application, after administration of the compound as shown in Formula I of the present application, the severity of chemotherapy-associated gastrointestinal side effects in the subject is relieved. In the present application, the relief may generally mean that the occurrence or development of the gastrointestinal side effects in the subject is delayed. In the present application, after administration of the compound as shown in Formula I, the symptoms of gastrointestinal side effects in the subject may be ameliorated.

In the present application, after administration of the compound as shown in Formula I, the symptoms of gastrointestinal side effects in the subject may be ameliorated by at least about 10%, e.g., by about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or more.

For example, in accordance with the criteria of NCI-CTCAE, after administration of the compound as shown in Formula I, the symptoms of gastrointestinal side effects (e.g., diarrhea, enteritis and/or constipation) in the subject may decrease from Grade 5 to Grade 4, from Grade 5 to Grade 3, from Grade 5 to Grade 2, from Grade 5 to Grade 1, from Grade 4 to Grade 3, from Grade 4 to Grade 2, from Grade 4 to Grade 1, from Grade 3 to Grade 2, from Grade 3 to Grade 1 or from Grade 2 to Grade 1.

Alternatively, in accordance with the method of Akinobu Kurita, after administration of the compound as shown in Formula I, the scoring of diarrhea in the subject may decrease from Grade 3 to Grade 2, from Grade 3 to Grade 1, from Grade 3 to Grade 0, from Grade 2 to Grade 1, from Grade 2 to Grade 0 or from Grade 1 to Grade 0.

In the present application, after administration of the compound as shown in Formula I, the symptoms of gastrointestinal side effects in the subject may be eliminated. However, the case that the gastrointestinal side effects recur or deteriorate again after stopping the administration of the compound as shown in Formula I may not be excluded.

### Therapeutic Method

The term “prevention” as used herein generally refers to the prevention of occurrence, recurrence, or spread of diseases or one or more symptoms thereof. In the context of the present application, the “prevention” may be interchangeably used with the “preventive treatment”. In some embodiments, the “prevention” generally refers to the treatment of providing a patient suffering from the diseases or disorders as described in the present application with the medicament in accordance with the present application with or without administration of other medicaments as described in the present application prior to the occurrence of any symptoms. In some embodiments, patients with a family history of a particular disease can be the candidates of a prevention program. In some embodiments, patients with a history of recurring symptoms are also potential objects of prevention.

The term “treatment” as used herein generally refers to eliminating or improving diseases or one or more symptoms associated with the diseases. In some embodiments, the treatment generally refers to eliminating or ameliorating a disease by administering one or more therapeutic agents to the patients suffering from the disease. In some embodiments, the “treatment” may be administering a medicament in the presence or absence of other therapeutic agent(s) after the occurrence of symptoms of a particular disease.

The term “subject” as used herein generally refers to a human or non-human animal (including mammals) in need of diagnosing, prognosing, improving, preventing, relieving and/or treating diseases, especially those in need of treatment, relief or prevention by using the compound as shown in Formula I. In some embodiments, the subject may comprise a cancer patient. For example, the cancer patient may have been, is being, and/or will be administered with the chemotherapy.

In some embodiments, the subject may be a human or a non-human mammal. The non-human mammals may include any mammalian species other than human, e.g., livestocks (e.g., cow, pig, sheep, chick, rabbit or horse), or rodents (e.g., rat and mouse), or primates (e.g., gorilla and monkey), or domestic animals (e.g., dog and cat). The “subject” may be male or female, and also may be at different ages.

The term “effective amount” as used herein generally refers to an amount of medicament capable of ameliorating or eliminating diseases or symptoms of the subject, or preventively inhibiting or prohibiting the occurrence of diseases or symptoms. The effective amount may be an amount of medicament capable of ameliorating one or more diseases or symptoms to some extent in the subject; an amount of medicament capable of partially or completely restoring one or more physiological or biochemical parameters associated with the causes of diseases or symptoms to normal; and/or an amount of medicament capable of reducing the possibility of the occurrence of diseases or symptoms.

In the present application, after the administration of the chemotherapy, for example, about 0.5 hrs, about 1 hr, about 2 hrs, about 3 hrs, about 4 hrs, about 5 hrs, about 6 hrs, about 7 hrs, about 8 hrs, about 9 hrs, about 10 hrs, about 11 hrs, about 12 hrs, about 13 hrs, about 14 hrs, about 15 hrs, about 16 hrs, about 17 hrs, about 18 hrs, about 19 hrs, about 20 hrs or longer before the administration of the chemotherapy, the medicament or the compound as shown in Formula I can be administered to prevent, relieve and/or treat the occurrence or deterioration of the gastrointestinal side effects. For example, the medicament or the compound as shown in Formula I is administered 0.5-12 hrs before the administration of the chemotherapy to prevent, relieve and/or treat the occurrence or deterioration of the gastrointestinal side effects.

In the present application, the administration site of the compound as shown in Formula I may be or not the occurrence site of cancer or the potential metastatic site of cancer. For example, the administration site may be or not the primary site of cancer. As another example, the administration site may be or not the metastasis site of cancer. For example, the metastasis site may comprise the occurrence site of cancer metastasis caused by lymphatic metastasis, vascular metastasis and/or implantation metastasis. In some embodiments, the metastasis site may comprise bone, brain, liver, stomach and/or lung. As another example, the administration site may be or not the recurrence site of cancer.

The compound as shown in Formula I of the present application may be administered in a manner known in the art, e.g., by injection (e.g., subcutaneous, intraperitoneal, intraarticular, intraarterial, intrathecal, intrasternal, intrathecal, intralesional, intracranial, intramuscular, intracutaneous and intravenous injection or infusion) or non-injection (e.g., oral, nasal, sublingual, vaginal, rectal, or topical administration). The compound as shown in Formula I of the present application may be administered in a form of a pharmaceutical combination or a kit. In some embodiments, the compound as shown in Formula I of the present application may be administered in the same administration route as that of the chemotherapy or in a different route.

In the present application, the medicament and/or the compound as shown in Formula I may be prepared for oral administration. For example, the medicament and/or the compound as shown in Formula I may be prepared as powders, tablets, granules, capsules, solution, emulsion and/or suspension.

In the present application, the medicament and/or the compound as shown in Formula I may be prepared for injection administration, e.g. administration through subcutaneous injection, intramuscular injection, intraperitoneal injection and/or intravenous injection. For example, the medicament and/or the compound as shown in Formula I may be prepared as injections.

In the present application, the dosage of the compound as shown in Formula I may be about 0.01-1000 mg/kg, for example, may be about 0.01-800 mg/kg, about 0.01-900 mg/kg, about 0.01-800 mg/kg, about 0.01-700 mg/kg, about 0.01-600 mg/kg, about 0.01-500 mg/kg, about 0.01-400 mg/kg, about 0.01-300 mg/kg, about 0.01-200 mg/kg, about 0.01-100 mg/kg, about 0.1-1000 mg/kg, about 1-1000 mg/kg, about 10-1000 mg/kg, about 50-1000 mg/kg, about 100-1000 mg/kg, about 0.1-800 mg/kg, about 1-600 mg/kg, about 10-500 mg/kg, about 10-400 mg/kg, about 15-300 mg/kg, about 50-250 mg/kg or about 50-200 mg/kg.

For example, the dosage of the compound as shown in Formula I in oral administration may be about 0.01-1000 mg/kg, for example, may be about 0.01-800 mg/kg, about 0.01-900 mg/kg, about 0.01-800 mg/kg, about 0.01-700 mg/kg, about 0.01-600 mg/kg, about 0.01-500 mg/kg, about 0.01-400 mg/kg, about 0.01-300 mg/kg, about 0.01-200 mg/kg, about 0.01-100 mg/kg, about 0.1-1000 mg/kg, about 1-1000 mg/kg, about 10-1000 mg/kg, about 50-1000 mg/kg, about 100-1000 mg/kg, about 0.1-800 mg/kg, about 1-600 mg/kg, about 10-500 mg/kg, about 10-400 mg/kg, about 15-300 mg/kg, about 50-250 mg/kg or about 50-200 mg/kg.

For example, the dosage of the compound as shown in Formula I in injection administration may be about 0.01-1000 mg/kg, for example, may be about 0.01-800 mg/kg, about 0.01-900 mg/kg, about 0.01-800 mg/kg, about 0.01-700 mg/kg, about 0.01-600 mg/kg, about 0.01-500 mg/kg, about 0.01-400 mg/kg, about 0.01-300 mg/kg, about 0.01-200 mg/kg, about 0.01-100 mg/kg, about 0.1-1000 mg/kg, about 1-1000 mg/kg, about 10-1000 mg/kg, about 50-1000 mg/kg, about 100-1000 mg/kg, about 0.1-800 mg/kg, about 1-600 mg/kg, about 10-500 mg/kg, about 10-400 mg/kg, about 15-300 mg/kg, about 50-250 mg/kg or about 50-200 mg/kg.

For example, the compound as shown in Formula I may be administered 0.5-24 h before the administration of the chemotherapeutic agent, may be administered orally, and the administration dosage may be about 50-150 mg/kg, the administration frequency may be once a week to once every 2 weeks (e.g., once every 7 days, once every 8 days, once every 9 days, once every 10 days, once every 11 days, once every 12 days, once every 13 days or once every 14 days).

For example, the compound as shown in Formula I may be administered 0.5-12 h before the administration of the chemotherapeutic agent, may be administered orally, and the administration dosage may be about 50-150 mg/kg, the administration cycle may be once a week to once every 2 weeks (e.g., once every 7 days, once every 8 days, once every 9 days, once every 10 days, once every 11 days, once every 12 days, once every 13 days or once every 14 days).

For example, the compound as shown in Formula I may be administered 0.5-24 h before the administration of the chemotherapeutic agent, may be administered by injection, and the administration dosage may be about 50-150 mg/kg, the administration frequency may be once a week to once every 2 weeks (e.g., once every 7 days, once every 8 days, once every 9 days, once every 10 days, once every 11 days, once every 12 days, once every 13 days or once every 14 days).

For example, the compound as shown in Formula I may be administered 0.5-12 h before the administration of the chemotherapeutic agent, may be administered by injection, and the administration dosage may be about 50-150 mg/kg, the administration cycle may be once a week to once every 2 weeks (e.g., once every 7 days, once every 8 days, once every 9 days, once every 10 days, once every 11 days, once every 12 days, once every 13 days or once every 14 days).

In the present application, the compound I and the pharmaceutically acceptable salt thereof (e.g., hydrochloride) can prevent, relieve and/or treat gastrointestinal side effects associated with fluorouracil in a subject. For example, the compound I and the pharmaceutically acceptable salt thereof may be administered 0.5-24 h before the administration of fluorouracil, the compound I and the pharmaceutically acceptable salt thereof may be administered by injection or orally, and the dosage of the compound I and the pharmaceutically acceptable salt thereof may be about 100 mg/kg-about 150 mg/kg.

For example, the compound I and the pharmaceutically acceptable salt thereof (e.g., hydrochloride) may be administered 0.5-12 h before the administration of fluorouracil, the compound I and the pharmaceutically acceptable salt thereof may be administered by intravenous injection, and the dosage of the compound I and the pharmaceutically acceptable salt thereof may be about 100 mg/kg-about 150 mg/kg.

For example, the compound I and the pharmaceutically acceptable salt thereof (e.g., hydrochloride) may be administered 0.5-12 h before the administration of fluorouracil, the compound I and the pharmaceutically acceptable salt thereof may be administered by intraperitoneal injection, and the dosage of the compound I and the pharmaceutically acceptable salt thereof may be about 100 mg/kg-about 150 mg/kg.

For example, the compound I and the pharmaceutically acceptable salt thereof (e.g., hydrochloride) may be administered 0.5-12 h before the administration of fluorouracil, the compound I and the pharmaceutically acceptable salt thereof may be administered by intramuscular injection, and the dosage of the compound I and the pharmaceutically acceptable salt thereof may be about 100 mg/kg-about 150 mg/kg.

For example, the compound I and the pharmaceutically acceptable salt thereof (e.g., hydrochloride) may be administered 0.5-12 h before the administration of fluorouracil, the compound I and the pharmaceutically acceptable salt thereof may be administered orally, and the dosage of the compound I and the pharmaceutically acceptable salt thereof may be about 100 mg/kg-about 150 mg/kg.

In the present application, the compound I and the pharmaceutically acceptable salt thereof (e.g., hydrochloride) can prevent, relieve and/or treat gastrointestinal side effects associated with oxaliplatin in a subject. For example, the compound I and the pharmaceutically acceptable salt thereof may be administered 0.5-24 h before the administration of oxaliplatin, the compound I and the pharmaceutically acceptable salt thereof may be administered by injection or orally, and the dosage of the compound I and the pharmaceutically acceptable salt thereof may be about 100 mg/kg-about 150 mg/kg.

For example, the compound I and the pharmaceutically acceptable salt thereof (e.g., hydrochloride) may be administered 0.5-12 h before the administration of oxaliplatin, the compound I and the pharmaceutically acceptable salt thereof may be administered by injection (e.g., intravenous injection or intraperitoneal injection), and the dosage of the compound I and the pharmaceutically acceptable salt thereof may be about 100 mg/kg-about 150 mg/kg.

In the present application, the compound I and the pharmaceutically acceptable salt thereof (e.g., hydrochloride) can prevent, relieve and/or treat gastrointestinal side effects associated with irinotecan in a subject. For example, the compound I and the pharmaceutically acceptable salt thereof may be administered 0.5-12 h before the administration of irinotecan, the compound I and the pharmaceutically acceptable salt thereof may be administered by injection or orally, and the dosage of the compound I and the pharmaceutically acceptable salt thereof may be about 100 mg/kg-about 150 mg/kg.

In the present application, the compound I and the pharmaceutically acceptable salt thereof (e.g., hydrochloride) can prevent, relieve and/or treat gastrointestinal side effects associated with the combined administration of fluorouracil and irinotecan in a subject. For example, the compound I and the pharmaceutically acceptable salt thereof may be administered 0.5-12 h before the administration of fluorouracil and irinotecan, the compound I and the pharmaceutically acceptable salt thereof may be administered by injection or orally, and the dosage of the compound I and the pharmaceutically acceptable salt thereof may be about 100 mg/kg-about 150 mg/kg.

In the present application, the compound I and the pharmaceutically acceptable salt thereof (e.g., hydrochloride) can prevent, relieve and/or treat gastrointestinal side effects associated with the combined administration of fluorouracil, oxaliplatin, irinotecan and tetrahydrofolic acid in a subject. For example, the compound I and the pharmaceutically acceptable salt thereof may be administered 0.5-12 h before the administration of fluorouracil, oxaliplatin, irinotecan and tetrahydrofolic acid, the compound I and the pharmaceutically acceptable salt thereof may be administered by injection or orally, and the dosage of the compound I and the pharmaceutically acceptable salt thereof may be about 100 mg/kg-about 150 mg/kg.

In the present application, the compound I and the pharmaceutically acceptable salt thereof can prevent, relieve and/or treat gastrointestinal side effects associated with gemcitabine in a subject.

In the present application, the compound I and the pharmaceutically acceptable salt thereof can prevent, relieve and/or treat gastrointestinal side effects associated with the combined administration of gemcitabine and carboplatin in a subject.

In the present application, the compound IV and the pharmaceutically acceptable salt thereof (e.g., hydrochloride) can prevent, relieve and/or treat gastrointestinal side effects associated with irinotecan in a subject. For example, the compound IV and the pharmaceutically acceptable salt thereof may be administered 0.5-12 h before the administration of irinotecan, the compound IV and the pharmaceutically acceptable salt thereof may be administered by injection, and the dosage of the compound I and the pharmaceutically acceptable salt thereof may be about 100 mg/kg-about 150 mg/kg.

In the present application, the compound IV and the pharmaceutically acceptable salt thereof (e.g., hydrochloride) can prevent, relieve and/or treat gastrointestinal side effects associated with fluorouracil in a subject. For example, the compound IV and the pharmaceutically acceptable salt thereof may be administered 0.5-12 h before the administration of fluorouracil, the compound IV and the pharmaceutically acceptable salt thereof may be administered by injection or orally, and the dosage of the compound IV and the pharmaceutically acceptable salt thereof may be about 100 mg/kg-about 150 mg/kg.

In the present application, the compound IV and the pharmaceutically acceptable salt thereof (e.g., hydrochloride) can prevent, relieve and/or treat gastrointestinal side effects associated with the combined administration of fluorouracil and irinotecan in a subject. For example, the compound IV and the pharmaceutically acceptable salt thereof may be administered 0.5-12 h before the administration of fluorouracil and irinotecan, the compound IV and the pharmaceutically acceptable salt thereof may be administered by injection or orally, and the dosage of the compound IV and the pharmaceutically acceptable salt thereof may be about 100 mg/kg-about 150 mg/kg.

In the present application, the compound IV and the pharmaceutically acceptable salt thereof (e.g., hydrochloride) can prevent, relieve and/or treat gastrointestinal side effects associated with the combined administration of fluorouracil, oxaliplatin, irinotecan and tetrahydrofolic acid in a subject. For example, the compound IV and the pharmaceutically acceptable salt thereof may be administered 0.5-24 h before the administration of fluorouracil, oxaliplatin, irinotecan and tetrahydrofolic acid, the compound IV and the pharmaceutically acceptable salt thereof may be administered by injection or orally, and the dosage of the compound IV and the pharmaceutically acceptable salt thereof may be about 100 mg/kg-about 150 mg/kg.

In the present application, the compound IV and the pharmaceutically acceptable salt thereof can prevent, relieve and/or treat gastrointestinal side effects associated with topotecan in a subject.

In the present application, the compound IV and the pharmaceutically acceptable salt thereof can prevent, relieve and/or treat gastrointestinal side effects associated with the combined administration of gemcitabine and carboplatin in a subject.

In the present application, the compound II and the pharmaceutically acceptable salt thereof can prevent, relieve and/or treat gastrointestinal side effects associated with fluorouracil in a subject. For example, the compound II and the pharmaceutically acceptable salt thereof may be administered 0.5-24 h before the administration of fluorouracil, the compound II and the pharmaceutically acceptable salt thereof may be administered by injection (e.g., intraperitoneal injection or intramuscular injection), and the dosage of the compound II and the pharmaceutically acceptable salt thereof may be about 50 mg/kg-about 150 mg/kg.

In the present application, the compound II and the pharmaceutically acceptable salt thereof can prevent, relieve and/or treat gastrointestinal side effects associated with the combined administration of fluorouracil, oxaliplatin, irinotecan and tetrahydrofolic acid in a subject. For example, the compound II and the pharmaceutically acceptable salt thereof may be administered 0.5-24 h before the combined administration of fluorouracil, oxaliplatin, irinotecan and tetrahydrofolic acid, the compound II and the pharmaceutically acceptable salt thereof may be administered orally or by injection, and the dosage of the compound II and the pharmaceutically acceptable salt thereof may be about 100 mg/kg-about 150 mg/kg.

In the present application, the compound II and the pharmaceutically acceptable salt thereof can prevent, relieve and/or treat gastrointestinal side effects associated with oxaliplatin in a subject. For example, the compound II and the pharmaceutically acceptable salt thereof may be administered 0.5-12 h before the administration of oxaliplatin, the compound II and the pharmaceutically acceptable salt thereof may be administered by injection, and the dosage of the compound II and the pharmaceutically acceptable salt thereof may be about 100 mg/kg-about 150 mg/kg.

In the present application, the compound II and the pharmaceutically acceptable salt thereof can prevent, relieve and/or treat gastrointestinal side effects associated with the combined administration of fluorouracil, oxaliplatin, irinotecan and tetrahydrofolic acid in a subject. For example, the compound II and the pharmaceutically acceptable salt thereof may be administered 0.5-24 h before the combined administration of fluorouracil, oxaliplatin, irinotecan and tetrahydrofolic acid, the compound II and the pharmaceutically acceptable salt thereof may be administered orally or by injection, and the dosage of the compound II and the pharmaceutically acceptable salt thereof may be about 100 mg/kg-about 150 mg/kg.

In the present application, the compound III and the pharmaceutically acceptable salt thereof can prevent, relieve and/or treat gastrointestinal side effects associated with oxaliplatin in a subject. For example, the compound III and the pharmaceutically acceptable salt thereof may be administered 0.5-12 h before the administration of oxaliplatin, the compound III and the pharmaceutically acceptable salt thereof may be administered orally or by injection, and the dosage of the compound III and the pharmaceutically acceptable salt thereof may be about 100 mg/kg-about 150 mg/kg.

In the present application, the compound III and the pharmaceutically acceptable salt thereof can prevent, relieve and/or treat gastrointestinal side effects associated with the combined administration of fluorouracil, oxaliplatin, irinotecan and tetrahydrofolic acid in a subject. For example, the compound III and the pharmaceutically acceptable salt thereof may be administered 0.5-24 h before the combined administration of fluorouracil, oxaliplatin, irinotecan and tetrahydrofolic acid, the compound III and the pharmaceutically acceptable salt thereof may be administered orally or by injection, and the dosage of the compound Ill and the pharmaceutically acceptable salt thereof may be about 150 mg/kg-about 200 mg/kg.

In the present application, the compound III and the pharmaceutically acceptable salt thereof can prevent, relieve and/or treat gastrointestinal side effects associated with fluorouracil in a subject. For example, the compound III and the pharmaceutically acceptable salt thereof may be administered 0.5-12 h before the administration of fluorouracil, the compound III and the pharmaceutically acceptable salt thereof may be administered orally or by injection, and the dosage of the compound III and the pharmaceutically acceptable salt thereof may be about 100 mg/kg-about 150 mg/kg.

In the present application, the medicament may further include one or more pharmaceutically acceptable carriers. The pharmaceutically acceptable carriers may include, but not limited to, e.g., pharmaceutically acceptable liquid, gel or solid carriers, aqueous medium, non-aqueous medium, antimicrobial substances, isotonic substances, buffer solution, antioxidants, anaesthetics, suspending agents/dispersing agents, chelating agents, emulsifiers, diluents, adjuvants, ingredients, non-toxic auxiliary substances, fillers, binders, disintegrants, buffer solution, preservatives, lubricants, flavoring agents, thickening agents, colorants, emulsifiers, other components well known in the art or various combinations of the above.

In another aspect, the present application provides a method of preventing, relieving and/or treating chemotherapy-associated gastrointestinal side effects with

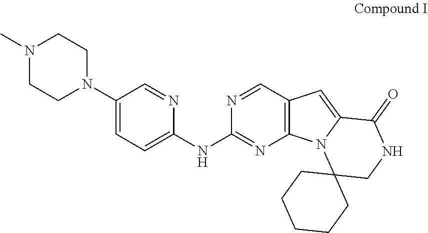 [SMILES: CN1CCN(c2ccc(Nc3ncc4cc5n(c4n3)C3(CCCCC3)CNC5=O)nc2)CC1]

or a pharmaceutically acceptable salt thereof. In some embodiments, the chemotherapy is fluorouracil, oxaliplatin, irinotecan (CPT-11), tetrahydrofolic acid, and a combination thereof. In some embodiments, the compound l is administered orally or by injection.

In another aspect, the present application provides a method of preventing, relieving and/or treating chemotherapy-associated gastrointestinal side effects with

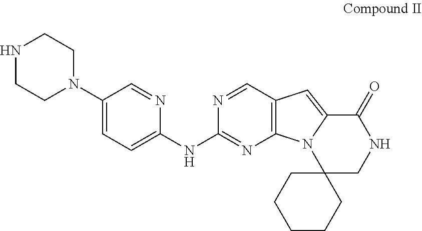 [SMILES: O=C1NCC2(CCCCC2)n2c1cc1cnc(Nc3ccc(N4CCNCC4)cn3)nc12]

or a pharmaceutically acceptable salt thereof. In some embodiments, the chemotherapy is fluorouracil, oxaliplatin, irinotecan (CPT-11), tetrahydrofolic acid, and a combination thereof. In some embodiments, the compound II is administered orally or by injection.

In another aspect, the present application provides a method of preventing, relieving and/or treating chemotherapy-associated gastrointestinal ide effects with

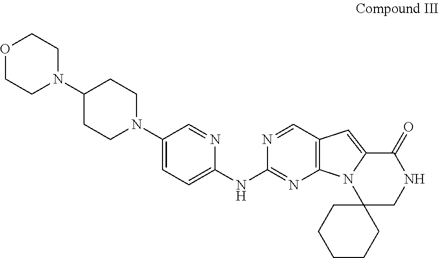 [SMILES: O=C1NCC2(CCCCC2)n2c1cc1cnc(Nc3ccc(N4CCC(N5CCOCC5)CC4)cn3)nc12]

or a pharmaceutically acceptable salt thereof. In some embodiments, the chemotherapy is fluorouracil, oxaliplatin, irinotecan (CPT-11), tetrahydrofolic acid, and a combination thereof. In some embodiments, the compound III is administered orally or by injection.

In another aspect, the present application provides a method of preventing, relieving and/or treating chemotherapy-associated gastrointestinal ide effects with

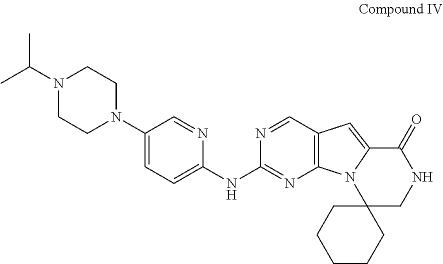 [SMILES: CC(C)N1CCN(c2ccc(Nc3ncc4cc5n(c4n3)C3(CCCCC3)CNC5=O)nc2)CC1]

or a pharmaceutically acceptable salt thereof. In some embodiments, the chemotherapy is fluorouracil, oxaliplatin, irinotecan (CPT-11), tetrahydrofolic acid, and a combination thereof. In some embodiments, the compound IV is administered orally or by injection.

In another aspect, the present application provides a method of preventing, relieving and/or treating chemotherapy-associated gastrointestinal side effects with the compound as shown in Formula I. In another aspect, the present application provides a use of the compound as shown in Formula I in preparing a medicament for preventing, relieving and/or treating chemotherapy-associated gastrointestinal side effects. In another aspect, the present application provides the compound as shown in Formula I, which is used for preventing, relieving and/or treating chemotherapy-associated gastrointestinal side effects. In some embodiments, the continuous administration time of the chemotherapy is less than 7 days. In some embodiments, the chemotherapy is administered orally or by injection.

In another aspect, the present application provides a method of preventing, relieving and/or treating chemotherapy-associated diarrhea with the compound as shown in Formula I. In another aspect, the present application provides a use of the compound as shown in Formula I in preparing a medicament for preventing, relieving and/or treating chemotherapy-associated diarrhea. In another aspect, the present application provides the compound as shown in Formula I, which is used for preventing, relieving and/or treating chemotherapy-associated diarrhea. In some embodiments, the continuous administration time of the chemotherapy is less than 7 days. In some embodiments, the chemotherapy is administered orally or by injection.

In another aspect, the present application provides a method of preventing, relieving and/or treating chemotherapy-associated constipation with the compound as shown in Formula I. In another aspect, the present application provides a use of the compound as shown in Formula I in preparing a medicament for preventing, relieving and/or treating chemotherapy-associated constipation. In another aspect, the present application provides the compound as shown in Formula I, which is used for preventing, relieving and/or treating chemotherapy-associated constipation. In some embodiments, the continuous administration time of the chemotherapy is less than 7 days. In some embodiments, the chemotherapy is administered orally or by injection.

Without being limited by any theory, the following examples are only for the purpose of illustrating the compounds, preparation methods and uses of the present application, and not intended to limit the invention scope of the present application.

### EXAMPLES

### Examples 1-201: Relieving Effect of the Compound as Shown in Formula I on Diarrhea Caused by a Chemotherapeutic Agent

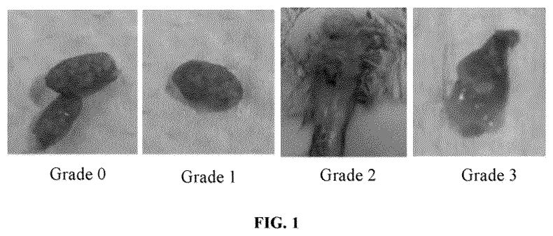

Construction of mouse animal models: Chemotherapeutic agent-caused diarrhea models of Balb/c mice were constructed according to the administration mode and frequency of the chemotherapeutic agent as shown in Table 3. After administration for several days, mice developed different extents of diarrhea symptoms (as shown in FIG. 1), which were similar to the conditions in human. Thus, chemotherapeutic agent-caused diarrhea models of mice are very good models to mimic diarrhea in human caused by the chemotherapeutic agent.

Balb/c mice were fed for 1 week, and then divided into groups. The experiment was divided into a control group, a chemotherapeutic agent group, and a group of the compound as shown in Formula I, each of which comprised 10 mice, to perform the administration experiment, for which the administration dosage, mode, time and frequency were shown in Table 3. The control group: being injected or intragastrically administered with the same solvent as that used in the group of the compound as shown in Formula I (the injection mode and time were shown in Table 3), and then being injected or intragastrically administered with the same solvent as that used in the chemotherapeutic agent group (the administration mode was shown in Table 3); the chemotherapeutic agent group: being injected or intragastrically administered with the same solvent as that used in the group of the compound as shown in Formula I (the administration mode and time were shown in Table 3), and then being injected or intragastrically administered with the chemotherapeutic agent (the type, administration mode and dosage were shown in Table 3); the group of the compound as shown in Formula I: being injected or intragastrically administered with the compound as shown in Formula I (the administration mode and time were shown in Table 3), and then being administered with the chemotherapeutic agent (the administration mode and dosage were shown in Table 3).

### Diarrhea Observation

The scoring of diarrhea can refer to the method of Akinobu Kurita (Cancer Chemother Pharmacol 2000; 46:211-20.), Grade 0: normal feces; Grade 1: mild diarrhea, the stools is slightly wet and soft; Grade 2: moderate diarrhea, loose stools and mild perianal contamination; Grade 3: severe diarrhea, watery stools and accompanied by severe perianal staining (see FIG. 1). It is regarded as effective remission when the diarrhea grading of mice in the group of the compound as shown in Formula I is reduced compared with the average diarrhea grading of mice in the chemotherapeutic agent group.

The diarrhea grading of mice was observed and recorded every day. At the end of the experiment, the number of mice in the group of the compound as shown in Formula I whose diarrhea grading was lower than that of mice in the chemotherapeutic agent group was recorded. Table 3 lists various animal experiment combinations of chemotherapeutic agents and the compound as shown in Formula I, as well as the corresponding experimental results. (Relative remission rate %=the number of mice in the group of the compound as shown in Formula I whose diarrhea has been effectively relieved/the total number of mice in the group of the compound as shown in Formula I*100%. The establishment rate of diarrhea model in the chemotherapeutic agent group was 50%-70%, that is, about 5-7 of 10 mice developed diarrhea during the experiment, and there were cases of individual mice death or not developing diarrhea.)

TABLE 3
Experimental Conditions and Results of Examples 1-201

| | | | | | | | Adminis- | | | | | | |
|---|---|---|---|---|---|---|---|---|---|---|---|---|---|
| | | | | | | | tration | | | | | | |
| | | | | | | | time | | | | | | |
| | | | | | | | (before the | | | | | Experi- | Relative |
| Exam- | Chemo- | Adminis- | | | | Compound | chemo- | | Adminis- | | | mental | remission |
| ple | therapeutic | tration | Dosage | | | as shown in | therapeutic | | tration | Dosage | | cycle | rate |
| No. | agent | mode | (mg/kg) | | Frequency | Formula I | agent) | | mode | (mg/kg) | Frequency | (days) | (%) |
| 1 | Fluorouracil | Intra- | 125 | | Single | Compound I | 0.5 | h | Intra- | 100 | Single | 10 | 70 |
| 2 | (5-Fu) | peritoneal | | | | (Trilaciclib) | 1 | h | venous | | | | 70 |
| 3 | | injection | | | | | 2 | h | injection | | | | 60 |
| 4 | | | | | | | 3 | h | | | | | 60 |
| 5 | | | | | | | 4 | h | | | | | 60 |
| 6 | | | | | | | 5 | h | | | | | 40 |
| 7 | | | | | | | 6 | h | | | | | 40 |
| 8 | | | | | | | 7 | h | | | | | 40 |
| 9 | | | | | | | 8 | h | | | | | 50 |
| 10 | | | | | | | 9 | h | | | | | 40 |
| 11 | | | | | | | 10 | h | | | | | 50 |
| 12 | | | | | | | 11 | h | | | | | 40 |
| 13 | | | | | | | 12 | h | | 12.5 | | | 30 |
| 14 | | | | | | | | | | 25 | | | 40 |
| 15 | | | | | | | | | | 50 | | | 60 |
| 16 | | | | | | | | | | 100 | | | 70 |
| 17 | | | | | | | | | | 150 | | | 50 |
| 18 | | | | | | | | | | 240 | | | 50 |
| 19 | | | | | | | 13 | h | | 100 | | | 60 |
| 20 | | | | | | | 14 | h | | | | | 60 |
| 21 | | | | | | | 16 | h | | | | | 60 |
| 22 | | | | | | | 18 | h | | | | | 50 |
| 23 | | | | | | | 20 | h | | | | | 50 |
| 24 | | | | | | | 22 | h | | | | | 40 |
| 25 | | | | | | | 24 | h | | | | | 40 |
| 26 | | | | | | | 48 | h | | | | | 0 |
| 27 | Fluorouracil | Intra- | 125 | | Single | Compound I | 0.5 | h | Intra- | 100 | Single | 10 | 50 |
| 28 | (5-Fu) | peritoneal | | | | hydrochloride | 2 | h | venous | | | | 70 |
| 29 | | injection | | | | (Trilaciclib | 4 | h | injection | | | | 70 |
| 30 | | | | | | dihydrochloride) | 12 | h | | | | | 60 |
| 31 | Fluorouracil | Intra- | 125 | | Single | Compound II | 0.5 | h | Intra- | 50 | Single | 10 | 30 |
| 32 | (5-Fu) | peritoneal | | | | | 4 | h | peritoneal | 100 | | | 60 |
| 33 | | injection | | | | | 12 | h | injection | 150 | | | 40 |
| 34 | Fluorouracil | Intra- | 175 | | Single | compound I | 0.5 | h | Intra- | 100 | Single | 10 | 40 |
| 35 | (5-Fu) | venous | | | | | 1 | h | peritoneal | | | | 40 |
| 36 | | injection | | | | | 2 | h | injection | | | | 30 |
| 37 | | | | | | | 4 | h | | | | | 30 |
| 38 | | | | | | | 6 | h | | | | | 50 |
| 39 | | | | | | | 8 | h | | | | | 60 |
| 40 | | | | | | | 10 | h | | | | | 50 |
| 41 | | | | | | | 12 | h | | | | | 40 |
| 42 | | | | | | | 14 | h | | | | | 30 |
| 43 | | | | | | | 16 | h | | | | | 40 |
| 44 | | | | | | | 18 | h | | | | | 50 |
| 45 | | | | | | | 20 | h | | | | | 50 |
| 46 | | | | | | | 24 | h | | | | | 40 |
| 47 | | | | | | | 48 | h | | | | | 0 |
| 48 | Fluorouracil | Intra- | 125 | | Single | Compound I | 0.5 | h | Intra- | 150 | Single | 10 | 30 |
| 49 | (5-Fu) | peritoneal | | | | hydrochloride | 2 | h | peritoneal | | | | 50 |
| 50 | | injection | | | | | 4 | h | injection | | | | 60 |
| 51 | | | | | | | 12 | h | | | | | 40 |
| 52 | Fluorouracil | Intra- | 175 | | Single | Compound I | 0.5 | h | Oral | 100 | Single | 10 | 40 |
| 53 | (5-Fu) | venous | | | | | 4 | h | adminis- | | | | 80 |
| 54 | | injection | | | | | 12 | h | tration | | | | 50 |
| 55 | | | | | | | 24 | h | | | | | 30 |
| 56 | Fluorouracil | Oral | 250 | | Single | Compound I | 0.5 | h | Intra- | 150 | Single | 8 | 60 |
| | (5-Fu) | adminis- | | | | | | | muscular | | | | |
| | | tration | | | | | | | injection | | | | |
| 57 | Fluorouracil | Intra- | 125 | | Single | Compound I | 8 | h | Intra- | 100 | Single | 10 | 40 |
| | (5-Fu) | muscular | | | | | | | peritoneal | | | | |
| | | injection | | | | | | | injection | | | | |
| 58 | Oxaliplatin | Intra- | 20 | | Single | Compound I | 0.5 | h | Intra- | 100 | Single | 8 | 40 |
| 59 | | peritoneal | | | | | 1 | h | peritoneal | | | | 30 |
| 60 | | injection | | | | | 2 | h | injection | | | | 40 |
| 61 | | | | | | | 3 | h | | | | | 50 |
| 62 | | | | | | | 4 | h | | | | | 50 |
| 63 | | | | | | | 5 | h | | | | | 30 |
| 64 | | | | | | | 6 | h | | | | | 40 |
| 65 | | | | | | | 7 | h | | | | | 60 |
| 66 | | | | | | | 8 | h | | | | | 60 |
| 67 | | | | | | | 9 | h | | | | | 50 |
| 68 | | | | | | | 10 | h | | | | | 70 |
| 69 | | | | | | | 11 | h | | | | | 70 |
| 70 | | | | | | | 12 | h | | | | | 50 |
| 71 | | | | | | | 14 | h | | | | | 60 |
| 72 | | | | | | | 16 | h | | | | | 50 |
| 73 | | | | | | | 18 | h | | | | | 50 |
| 74 | | | | | | | 20 | h | | | | | 70 |
| 75 | | | | | | | 22 | h | | | | | 40 |
| 76 | | | | | | | 24 | h | | | | | 40 |
| 77 | | | | | | | 48 | h | | | | | 0 |
| 78 | Oxaliplatin | Intra- | 20 | | Single | Compound I | 0.5 | h | Intra- | 150 | Single | 8 | 20 |
| 79 | | peritoneal | | | | hydrochloride | 6 | h | peritoneal | | | | 60 |
| 80 | | injection | | | | | 12 | h | injection | | | | 40 |
| 81 | Oxaliplatin | Intra- | 17.5 | | Single | Compound I | 0.5 | h | Intra- | 150 | Single | 8 | 20 |
| 82 | | venous | | | | | 4 | h | venous | | | | 30 |
| 83 | | injection | | | | | 8 | h | injection | | | | 30 |
| 84 | | | | | | | 12 | h | | | | | 40 |
| 85 | | | | | | | 16 | h | | | | | 40 |
| 86 | | | | | | | 20 | h | | | | | 40 |
| 87 | | | | | | | 24 | h | | | | | 50 |
| 88 | | | | | | | 48 | h | | | | | 0 |
| 89 | Oxaliplatin | Intra- | 17.5 | | Single | Compound III | 1 | h | Intra- | 100 | Single | 8 | 40 |
| 90 | | venous | | | | | 4 | h | peritoneal | | | | 70 |
| 91 | | injection | | | | | 8 | h | injection | | | | 40 |
| 92 | Irinotecan | Intra- | 240 | | Single | Compound I | 0.5 | h | Intra- | 100 | Single | 7 | 50 |
| 93 | (CPT-11) | peritoneal | | | | | 1 | h | venous | | | | 50 |
| 94 | | injection | | | | | 2 | h | injection | | | | 50 |
| 95 | | | | | | | 3 | h | | | | | 40 |
| 96 | | | | | | | 4 | h | | | | | 50 |
| 97 | | | | | | | 5 | h | | | | | 40 |
| 98 | | | | | | | 6 | h | | | | | 40 |
| 99 | | | | | | | 7 | h | | | | | 70 |
| 100 | | | | | | | 8 | h | | | | | 70 |
| 101 | | | | | | | 9 | h | | | | | 60 |
| 102 | | | | | | | 10 | h | | | | | 40 |
| 103 | | | | | | | 11 | h | | | | | 60 |
| 104 | | | | | | | 12 | h | | | | | 60 |
| 105 | | | | | | | 14 | h | | | | | 50 |
| 106 | | | | | | | 16 | h | | | | | 70 |
| 107 | | | | | | | 18 | h | | | | | 70 |
| 108 | | | | | | | 20 | h | | | | | 40 |
| 109 | | | | | | | 22 | h | | | | | 40 |
| 110 | | | | | | | 24 | h | | | | | 30 |
| 111 | | | | | | | 48 | h | | | | | 0 |
| 112 | Irinotecan | Intra- | 240 | | Single | Compound IV | 0.5 | h | Intra- | 100 | Single | 11 | 60 |
| 113 | (CPT-11) | peritoneal | | | | (Lerociclib) | 4 | h | peritoneal | | | | 50 |
| 114 | | injection | | | | | 8 | h | injection | | | | 30 |
| 115 | Irinotecan | Intra- | 240 | | Single | Compound IV | 0.5 | h | Intra- | 100 | Single | 11 | 40 |
| 116 | (CPT-11) | peritoneal | | | | hydrochloride | 4 | h | peritoneal | | | | 60 |
| 117 | | injection | | | | (Lerociclibdi- | 12 | h | injection | | | | 20 |
| | | | | | | hydrochloride) | | | | | | | |
| 118 | Irinotecan | Oral | 300 | | Single | Compound I | 12 | h | Intra- | 100 | Single | 11 | 50 |
| | (CPT-11) | adminis- | | | | | | | peritoneal | | | | |
| | | tration | | | | | | | injection | | | | |
| 119 | Irinotecan | Intra- | 250 | | Single | Compound I | 1 | h | Intra- | 100 | Single | 7 | 70 |
| | (CPT-11) | muscular | | | | | | | muscular | | | | |
| | | injection | | | | | | | injection | | | | |
| 120 | Irinotecan | Intra- | 200 | | Single | Compound I | 0. | h | Intra- | 100 | Single | 7 | 70 |
| | (CPT-11) | venous | | | | | | | venous | | | | |
| | | injection | | | | | | | injection | | | | |
| 121 | Fluorouracil | Intra- | 100 | (F) | Single | Compound I | 0.5 | h | Intra- | 100 | Single | 10 | 70 |
| 122 | and | venous | 200 | (I) | | | 4 | h | venous | | | | 40 |
| 123 | irinotecan | injection | | | | | 8 | h | injection | | | | 50 |
| 124 | | | | | | | 12 | h | | | | | 50 |
| 125 | | | | | | | 24 | h | | | | | 30 |
| 126 | | | | | | | 48 | h | | | | | 0 |
| 127 | Fluorouracil | Intra- | 100 | (F) | Single | Compound I | 1 | h | Intra- | 100 | Single | 8 | 40 |
| 128 | and | peritoneal | 200 | (I) | | | 12 | h | peritoneal | | | | 30 |
| | irinotecan | injection | | | | | | | injection | | | | |
| 129 | Fluorouracil | Intra- | 100 | (F) | Single | Compound I | 0.5 | h | Oral | 150 | Single | 8 | 40 |
| 130 | and | peritoneal | 200 | (I) | | | 12 | h | adminis- | | | | 40 |
| 131 | irinotecan | injection | | | | | 24 | h | tration | | | | 30 |
| 132 | Fluorouracil | Intra- | 100 | (F) | Single | Compound I | 0.5 | h | Intra- | 150 | Single | 8 | 30 |
| 133 | and | peritoneal | 200 | (I) | | hydrochloride | 6 | h | peritoneal | | | | 60 |
| 134 | irinotecan | injection | | | | | 12 | h | injection | | | | 50 |
| 135 | Fluorouracil | Intra- | 100 | (F) | Single | Compound IV | 0.5 | h | Oral | 100 | Single | 8 | 30 |
| 136 | and | peritoneal | 200 | (I) | | | 12 | h | adminis- | | | | 50 |
| 137 | irinotecan | injection | | | | | 24 | h | tration | | | | 40 |
| 138 | Fluorouracil | Intra- | 50 | (F) | Single | Compound I | 0.5 | h | Intra- | 100 | Single | 10 | 50 |
| 139 | and | peritoneal | 5 | (O) | | | 4 | h | peritoneal | | | | 40 |
| 140 | oxaliplatin | injection | 50 | (I) | | | 8 | h | injection | | | | 40 |
| 141 | and | | | | | | 12 | h | | | | | 50 |
| 142 | irinotecan | | | | | | 24 | h | | | | | 50 |
| 143 | | | | | | | 48 | h | | | | | 0 |
| 144 | Fluorouracil | Intra- | 50 | (F) | Single | Compound I | 0.5 | h | Intra- | 100 | Single | 10 | 50 |
| 145 | and | venous | 5 | (O) | | | 1 | h | venous | | | | 40 |
| 146 | oxaliplatin | injection | 50 | (I) | | | 2 | h | injection | | | | 40 |
| 147 | and | | 90 | (L) | | | 3 | h | | | | | 30 |
| 148 | irinotecan | | | | | | 4 | h | | | | | 40 |
| 149 | and | | | | | | 6 | h | | | | | 40 |
| 150 | tetrahydro- | | | | | | 8 | h | | | | | 30 |
| 151 | folic acid | | | | | | 10 | h | | | | | 30 |
| 152 | | | | | | | 12 | h | | | | | 30 |
| 153 | | | | | | | 14 | h | | | | | 50 |
| 154 | | | | | | | 16 | h | | | | | 50 |
| 155 | | | | | | | 18 | h | | | | | 60 |
| 156 | | | | | | | 20 | h | | | | | 60 |
| 157 | | | | | | | 24 | h | | | | | 30 |
| 158 | | | | | | | 48 | h | | | | | 0 |
| 159 | Fluorouracil | Intra- | 50 | (F) | Single | Compound I | 0.5 | h | Intra- | 100 | Single | 7 | 50 |
| 160 | and | peritoneal | 5 | (O) | | | 24 | h | peritoneal | | | | 20 |
| | oxaliplatin | injection | 50 | (I) | | | | | injection | | | | |
| | and | | 90 | (L) | | | | | | | | | |
| | irinotecan | | | | | | | | | | | | |
| | and | | | | | | | | | | | | |
| | tetrahydro- | | | | | | | | | | | | |
| | folic acid | | | | | | | | | | | | |
| 161 | Fluorouracil | Intra- | 50 | (F) | Single | Compound I | 0.5 | h | Intra- | 150 | Single | 7 | 70 |
| 162 | and | peritoneal | 5 | (O) | | hydrochloride | 12 | h | peritoneal | | | | 40 |
| | oxaliplatin | injection | 50 | (I) | | | | | injection | | | | |
| | and | | 90 | (L) | | | | | | | | | |
| | irinotecan | | | | | | | | | | | | |
| | and | | | | | | | | | | | | |
| | tetrahydro- | | | | | | | | | | | | |
| | folic acid | | | | | | | | | | | | |
| 163 | Fluorouracil | Intra- | 50 | (F) | Single | Compound I | 0.5 | h | Oral | 150 | Single | 7 | 40 |
| 164 | and | peritoneal | 5 | (O) | | | 4 | h | adminis- | | | | 60 |
| 165 | oxaliplatin | injection | 50 | (I) | | | 8 | h | tration | | | | 80 |
| 166 | and | | 90 | (L) | | | 12 | h | | | | | 40 |
| 167 | irinotecan | | | | | | 24 | h | | | | | 20 |
| | and | | | | | | | | | | | | |
| | tetrahydro- | | | | | | | | | | | | |
| | folic acid | | | | | | | | | | | | |
| 168 | Fluorouracil | Intra- | 50 | (F) | Single | Compound I | 0.5 | h | Oral | 150 | Single | 7 | 40 |
| 169 | and | peritoneal | 5 | (O) | | | 4 | h | adminis- | | | | 60 |
| 170 | oxaliplatin | injection | 50 | (I) | | | 8 | h | tration | | | | 80 |
| 171 | and | | 90 | (L) | | | 12 | h | | | | | 40 |
| 172 | irinotecan | | | | | | 24 | h | | | | | 20 |
| | and | | | | | | | | | | | | |
| | tetrahydro- | | | | | | | | | | | | |
| | folic acid | | | | | | | | | | | | |
| 173 | Fluorouracil | Intra- | 50 | (F) | Single | Compound II | 0.5 | h | Oral | 100 | Single | 7 | 30 |
| 174 | and | peritoneal | 5 | (O) | | | 4 | h | adminis- | | | | 30 |
| 175 | oxaliplatin | injection | 50 | (I) | | | 8 | h | tration | | | | 40 |
| 176 | and | | 90 | (L) | | | 12 | h | | | | | 30 |
| 177 | irinotecan | | | | | | | | | | | | |
| | and | | | | | | 24 | h | | | | | 30 |
| | tetrahydro- | | | | | | | | | | | | |
| | folic acid | | | | | | | | | | | | |
| 178 | Fluorouracil | Intra- | 50 | (F) | Single | Compound III | 0.5 | h | Oral | 150 | Single | 7 | 30 |
| 179 | and | venous | 5 | (O) | | | 4 | h | adminis- | | | | 50 |
| 180 | oxaliplatin | injection | 50 | (I) | | | 8 | h | tration | | | | 40 |
| 181 | and | | 90 | (L) | | | 12 | h | | | | | 40 |
| 182 | irinotecan | | | | | | 24 | h | | | | | 20 |
| | and | | | | | | | | | | | | |
| | tetrahydro- | | | | | | | | | | | | |
| | folic acid | | | | | | | | | | | | |
| 183 | Fluorouracil | Intra- | 50 | (F) | Single | Compound IV | 0.5 | h | Intra- | 100 | Single | 7 | 80 |
| 184 | and | venous | 5 | (O) | | | 4 | h | peritoneal | | | | 60 |
| 185 | oxaliplatin | injection | 50 | (I) | | | 8 | h | injection | | | | 50 |
| 186 | and | | 90 | (L) | | | 12 | h | | | | | 50 |
| 187 | irinotecan | | | | | | 24 | h | | | | | 40 |
| | and | | | | | | | | | | | | |
| | tetrahydro- | | | | | | | | | | | | |
| | folic acid | | | | | | | | | | | | |
| 188 | Fluorouracil | Intra- | 50 | (F) | Single | Compound IV | 0.5 | h | Intra- | 100 | Single | 7 | 70 |
| 189 | and | venous | 5 | (O) | | hydrochloride | 12 | h | peritoneal | | | | 50 |
| | oxaliplatin | injection | 50 | (I) | | | | | injection | | | | |
| | and | | 90 | (L) | | | | | | | | | |
| | irinotecan | | | | | | | | | | | | |
| | and | | | | | | | | | | | | |
| | tetrahydro- | | | | | | | | | | | | |
| | folic acid | | | | | | | | | | | | |
| 190 | Fluorouracil | Oral | 100 | (F) | Single | Compound I | 1 | h | sub- | 100 | Single | 7 | 60 |
| 191 | and | adminis- | 10 | (O) | | | 2 | h | cutaneous | | | | 50 |
| 192 | oxaliplatin | tration | 200 | (I) | | | 4 | h | injection | | | | 30 |
| 193 | and | | 100 | (L) | | | 6 | h | | | | | 30 |
| | irinotecan | | | | | | | | | | | | |
| | and | | | | | | | | | | | | |
| | tetrahydro- | | | | | | | | | | | | |
| | folic acid | | | | | | | | | | | | |
| 194 | Topotecan | Intra- | 10 | | Single | Compound I | 4 | h | Oral | 100 | Single | 14 | 50 |
| | | peritoneal | | | | hydrochloride | | | adminis- | | | | |
| | | injection | | | | | | | tration | | | | |
| 195 | Topotecan | Intra- | 20 | | Single | Compound IV | 0.5 | h | Oral | 100 | Single | 14 | 60 |
| | | venous | | | | | | | adminis- | | | | |
| | | injection | | | | | | | tration | | | | |
| 196 | Gemcitabine | Intra- | 120 | | Single | Compound I | 12 | h | Oral | 100 | Single | 8 | 50 |
| | (GEM) | peritoneal | | | | | | | adminis- | | | | |
| | | injection | | | | | | | tration | | | | |
| 197 | Gemcitabine | Intra- | 120 | | Single | Compound II | 24 | h | Oral | 100 | Single | 8 | 40 |
| | (GEM) | venous | | | | | | | adminis- | | | | |
| | | injection | | | | | | | tration | | | | |
| 198 | Etoposide | Intra- | 60 | (E) | Single | Compound II | 2 | h | Oral | 100 | Single | 10 | 50 |
| | and | peritoneal | 5 | (C) | | | | | adminis- | | | | |
| | carboplatin | injection | | | | | | | tration | | | | |
| 199 | Etoposide | Intra- | 60 | (E) | Single | Compound III | 0.5 | h | Oral | 100 | Single | 10 | 60 |
| | and | peritoneal | 5 | (C) | | | | | adminis- | | | | |
| | carboplatin | injection | | | | | | | tration | | | | |
| 200 | Gemcitabine | Intra- | 120 | | Single | Compound I | 4 | h | Oral | 100 | Single | 8 | 70 |
| | and | peritoneal | | | | hydrochloride | | | adminis- | | | | |
| | carboplatin | injection | | | | | | | tration | | | | |
| 201 | Gemcitabine | Intra- | 120 | (G) | Single | Compound IV | 24 | h | Oral | 100 | Single | 8 | 40 |
| | and | venous | 5 | (C) | | hydrochloride | | | adminis- | | | | |
| | carboplatin | injection | | | | | | | tration | | | | |

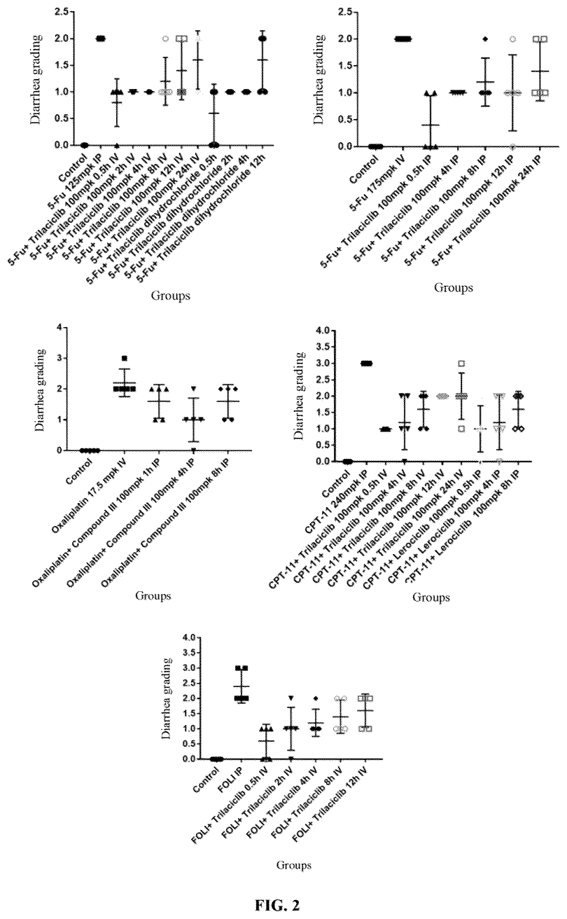

FIG. 1 shows typical photographs showing different diarrhea gradings of mice in the chemotherapeutic agent group in Table 3. FIG. 2 shows partial diarrhea grading results of the control group, the chemotherapeutic agent group, and the group of the compound as shown in Formula I in Table 3.

It can be seen from the results in Table 3 and FIG. 2 that, the diarrhea gradings in the groups of compounds I, II, IV and III or their hydrochlorides were relieved to different degrees compared with that in the chemotherapeutic agent group. Therefore, the administration of compounds I, II, IV and III or their hydrochlorides before the chemotherapeutic agent can effectively relieve diarrhea caused by the chemotherapeutic agent.

### Examples 202-364: Relieving Effect of the Compound as Shown in Formula I on Constipation Caused by a Chemotherapeutic Agent

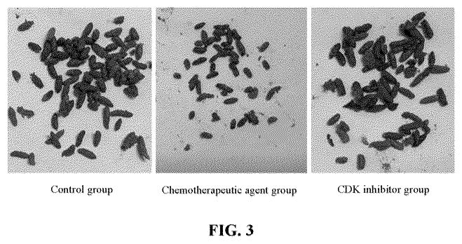

Construction of mouse animal models: Chemotherapeutic agent-caused constipation models of Balb/c mice were constructed according to the administration mode and frequency of the chemotherapeutic agent as shown in Table 4. After administration for several days, mice showed decreased stool volume (as shown in FIG. 3), which was consistent with the symptoms of constipation caused by a chemotherapeutic agent in human. Thus, chemotherapeutic agent-caused constipation models of mice are very good models to mimic constipation caused by the chemotherapeutic agent.

Balb/c mice were fed for 1 week, and then divided into groups. The experiment was divided into a control group, a chemotherapeutic agent group, and a group of the compound as shown in Formula I, each of which comprised 10 mice, to perform the administration experiment, for which the administration dosage, mode, time and frequency were shown in Table 4. The control group: being injected or intragastrically administered with the same solvent as that used in the group of the compound as shown in Formula I (the administration mode and time were shown in Table 4), and then being injected or intragastrically administered with the same solvent as that used in the chemotherapeutic agent group (the administration mode was shown in Table 4); the chemotherapeutic agent group: being injected or intragastrically administered with the same solvent as that used in the group of the compound as shown in Formula I (the administration mode and time was shown in Table 4), and then being injected or intragastrically administered with the chemotherapeutic agent (the type, administration mode and dosage were shown in Table 4); the group of the compound as shown in Formula I: being injected or intragastrically administered with the compound as shown in Formula I (the administration mode and time was shown in Table 4), and then being administered with the chemotherapeutic agent (the administration mode and dosage were shown in Table 4).

### Constipation Observation

The feces of mice within 3 hours were collected, which were observed for shape and weighed with an electronic balance. The feces reduction rate was calculated with reference to the method of Ji Eun Kim (Lab Anim Res. 2016 December; 32(4): 231-240.). Feces reduction rate (%)=(the control group−the chemotherapeutic agent group or the group of the compound as shown in Formula I)/the control group*100%

It is regarded as effective remission when the feces reduction rate of mice in the group of the compound as shown in Formula I is reduced compared with that in the chemotherapeutic agent group.

The feces amount of mice within 3 hours was observed and recorded every day. At the end of the experiment, the number of mice in the group of the compound as shown in Formula I whose feces reduction rate was lower than that of mice in the chemotherapeutic agent group was recorded. Table 4 lists various animal experiment combinations of chemotherapeutic agents and the compound as shown in Formula I, as well as the corresponding experimental results. (Relative remission rate %=the number of mice in the group of the compound as shown in Formula I which have been effectively relieved/the total number of mice in the group of the compound as shown in Formula I*100%. The establishment rate of constipation model in the chemotherapeutic agent group was 30%-60%, that is, about 3-6 of 10 mice showed reduced feces amount during the experiment, and there were cases of individual mice death or with no changes or an increasement in the feces amount.)

TABLE 4
Experimental Conditions and Results of Examples 202-364

| | | | | | | | Adminis- | | | | | | |
|---|---|---|---|---|---|---|---|---|---|---|---|---|---|
| | | | | | | | tration | | | | | | |
| | | | | | | | time | | | | | | |
| | | | | | | | (before | | | | | Experi- | Relative |
| Exam- | Chemo- | Adminis- | | | | Compound | the chemo- | | Adminis- | | | mental | remission |
| ple | therapeutic | tration | Dosage | | | as shown in | therapeutic | | tration | Dosage | | cycle | rate |
| No. | agent | mode | (mg/kg) | | Frequency | Formula I | agent) | | mode | (mg/kg) | Frequency | (days) | (%) |
| 202 | Fluorouracil | Intra- | 175 | | Single | Compound I | 0.5 | h | Intra- | 100 | 8 | 10 | 40 |
| 203 | (5-Fu) | peritoneal | | | | | 1 | h | venous | | | | 30 |
| 204 | | injection | | | | | 2 | h | injection | | | | 40 |
| 205 | | | | | | | 3 | h | | | | | 40 |
| 206 | | | | | | | 4 | h | | | | | 30 |
| 207 | | | | | | | 5 | h | | | | | 20 |
| 208 | | | | | | | 6 | h | | | | | 50 |
| 209 | | | | | | | 7 | h | | | | | 50 |
| 210 | | | | | | | 8 | h | | | | | 40 |
| 211 | | | | | | | 9 | h | | | | | 40 |
| 212 | | | | | | | 10 | h | | | | | 30 |
| 213 | | | | | | | 11 | h | | | | | 50 |
| 214 | | | | | | | 12 | h | | 12.5 | | | 20 |
| 215 | | | | | | | | | | 25 | | | 30 |
| 216 | | | | | | | | | | 50 | | | 30 |
| 217 | | | | | | | | | | 100 | | | 40 |
| 218 | | | | | | | | | | 150 | | | 60 |
| 219 | | | | | | | | | | 240 | | | 60 |
| 220 | | | | | | | 13 | h | | 100 | | | 40 |
| 221 | | | | | | | 14 | h | | | | | 40 |
| 222 | | | | | | | 16 | h | | | | | 50 |
| 223 | | | | | | | 18 | h | | | | | 60 |
| 224 | | | | | | | 20 | h | | | | | 60 |
| 225 | | | | | | | 22 | h | | | | | 30 |
| 226 | | | | | | | 24 | h | | | | | 30 |
| 227 | | | | | | | 48 | h | | | | | 0 |
| 228 | Fluorouracil | Intra- | 250 | | Single | Compound I | 0.5 | h | Intra- | 100 | Single | 10 | 30 |
| 229 | (5-Fu) | venous | | | | | 1 | h | peritoneal | | | | 40 |
| 230 | | injection | | | | | 2 | h | injection | | | | 40 |
| 231 | | | | | | | 4 | h | | | | | 60 |
| 232 | | | | | | | 6 | h | | | | | 60 |
| 233 | | | | | | | 8 | h | | | | | 50 |
| 234 | | | | | | | 10 | h | | | | | 50 |
| 235 | | | | | | | 12 | h | | | | | 70 |
| 236 | | | | | | | 14 | h | | | | | 40 |
| 237 | | | | | | | 16 | h | | | | | 40 |
| 238 | | | | | | | 18 | h | | | | | 50 |
| 239 | | | | | | | 20 | h | | | | | 50 |
| 240 | | | | | | | 24 | h | | | | | 50 |
| 241 | | | | | | | 48 | h | | | | | 0 |
| 242 | Fluorouracil | Intra- | 250 | | Single | Compound I | 0.5 | h | Intra- | 150 | Single | 10 | 30 |
| 243 | (5-Fu) | venous | | | | hydrochloride | 4 | h | peritoneal | | | | 50 |
| 244 | | injection | | | | | 8 | h | injection | | | | 70 |
| 245 | | | | | | | 12 | h | | | | | 60 |
| 246 | Fluorouracil | Intra- | 250 | | Single | Compound IV | 0.5 | h | Oral | 100 | Single | 10 | 30 |
| 247 | (5-Fu) | venous | | | | | 4 | h | adminis- | | | | 60 |
| 248 | | injection | | | | | 12 | h | tration | | | | 20 |
| 249 | Fluorouracil | Oral | 300 | | Single | Compound I | 0.5 | h | Intra- | 100 | Single | 7 | 60 |
| | (5-Fu) | adminis- | | | | | | | muscular | | | | |
| | | tration | | | | | | | injection | | | | |
| 250 | Fluorouracil | Intra- | 175 | | Single | Compound I | 8 | h | Oral | 150 | Single | 7 | 50 |
| | (5-Fu) | muscular | | | | | | | adminis- | | | | |
| | | injection | | | | | | | tration | | | | |
| 251 | Oxaliplatin | Intra- | 25 | | Single | Compound I | 0.5 | h | Intra- | 100 | Single | 10 | 20 |
| 252 | | peritoneal | | | | | 1 | h | peritoneal | | | | 20 |
| 253 | | injection | | | | | 2 | h | injection | | | | 40 |
| 254 | | | | | | | 3 | h | | | | | 40 |
| 255 | | | | | | | 4 | h | | | | | 50 |
| 256 | | | | | | | 5 | h | | | | | 50 |
| 257 | | | | | | | 6 | h | | | | | 60 |
| 258 | | | | | | | 7 | h | | | | | 60 |
| 259 | | | | | | | 8 | h | | | | | 30 |
| 260 | | | | | | | 9 | h | | | | | 40 |
| 261 | | | | | | | 10 | h | | | | | 40 |
| 262 | | | | | | | 11 | h | | | | | 50 |
| 263 | | | | | | | 12 | h | | | | | 50 |
| 264 | | | | | | | 14 | h | | | | | 30 |
| 265 | | | | | | | 16 | h | | | | | 40 |
| 266 | | | | | | | 18 | h | | | | | 60 |
| 267 | | | | | | | 20 | h | | | | | 30 |
| 268 | | | | | | | 22 | h | | | | | 30 |
| 269 | | | | | | | 24 | h | | | | | 40 |
| 270 | | | | | | | 48 | h | | | | | 0 |
| 271 | Oxaliplatin | Intra- | 25 | | Single | Compound I | 0.5 | h | Intra- | 150 | Single | 10 | 30 |
| 272 | | peritoneal | | | | hydrochloride | 4 | h | peritoneal | | | | 40 |
| 273 | | injection | | | | | 8 | h | injection | | | | 50 |
| 274 | | | | | | | 12 | h | | | | | 50 |
| 275 | Oxaliplatin | Intra- | 25 | | Single | Compound I | 0.5 | h | Intra- | 150 | Single | 7 | 30 |
| 276 | | venous | | | | | 4 | h | venous | | | | 50 |
| 277 | | injection | | | | | 8 | h | injection | | | | 50 |
| 278 | | | | | | | 12 | h | | | | | 40 |
| 279 | | | | | | | 16 | h | | | | | 40 |
| 280 | | | | | | | 20 | h | | | | | 60 |
| 281 | | | | | | | 24 | h | | | | | 60 |
| 282 | | | | | | | 48 | h | | | | | 0 |
| 283 | Oxaliplatin | Intra- | 25 | | Single | Compound II | 1 | h | Intra- | 100 | Single | 7 | 50 |
| 284 | | venous | | | | | 6 | h | muscular | | | | 30 |
| 285 | | injection | | | | | 12 | h | injection | | | | 30 |
| 286 | Irinotecan | Intra- | 260 | | Single | Compound I | 0.5 | h | Intra- | 150 | Single | 8 | 40 |
| 287 | (CPT-11) | peritoneal | | | | | 1 | h | venous | | | | 40 |
| 288 | | injection | | | | | 2 | h | injection | | | | 50 |
| 289 | | | | | | | 3 | h | | | | | 50 |
| 290 | | | | | | | 4 | h | | | | | 40 |
| 291 | | | | | | | 5 | h | | | | | 30 |
| 292 | | | | | | | 6 | h | | | | | 40 |
| 293 | | | | | | | 7 | h | | | | | 50 |
| 294 | | | | | | | 8 | h | | | | | 60 |
| 295 | | | | | | | 9 | h | | | | | 70 |
| 296 | | | | | | | 10 | h | | | | | 50 |
| 297 | | | | | | | 11 | h | | | | | 50 |
| 298 | | | | | | | 12 | h | | | | | 40 |
| 299 | | | | | | | 14 | h | | | | | 40 |
| 300 | | | | | | | 16 | h | | | | | 40 |
| 301 | | | | | | | 18 | h | | | | | 50 |
| 302 | | | | | | | 20 | h | | | | | 50 |
| 303 | | | | | | | 22 | h | | | | | 40 |
| 304 | | | | | | | 24 | h | | | | | 40 |
| 305 | | | | | | | 48 | h | | | | | 0 |
| 306 | Irinotecan | Oral | 300 | | Single | Compound I | 24 | h | Intra- | 100 | Single | 10 | 60 |
| | (CPT-11) | adminis- | | | | | | | peritoneal | | | | |
| | | tration | | | | | | | injection | | | | |
| 307 | Irinotecan | Intra- | 260 | | Single | Compound I | 1 | h | Intra- | 100 | Single | 7 | 60 |
| | (CPT-11) | muscular | | | | | | | muscular | | | | |
| | | injection | | | | | | | injection | | | | |
| 308 | Irinotecan | Intra- | 260 | | Single | Compound I | 12 | h | Oral | 100 | Single | 7 | 50 |
| | (CPT-11) | venous | | | | | | | adminis- | | | | |
| | | injection | | | | | | | tration | | | | |
| 309 | Fluorouracil | Intra- | 125 | (F) | Single | Compound I | 0.5 | h | Intra- | 100 | Single | 10 | 40 |
| 310 | and | venous | 240 | (I) | | | 4 | h | venous | | | | 60 |
| 311 | irinotecan | injection | | | | | 8 | h | injection | | | | 60 |
| 312 | | | | | | | 12 | h | | | | | 70 |
| 313 | | | | | | | 24 | h | | | | | 50 |
| 314 | | | | | | | 48 | h | | | | | 0 |
| 315 | Fluorouracil | Intra- | 125 | (F) | Single | Compound I | 0.5 | h | Intra- | 150 | Single | 10 | 20 |
| 316 | and | peritoneal | 240 | (I) | | hydrochloride | 4 | h | peritoneal | | | | 60 |
| 317 | irinotecan | injection | | | | | 12 | h | injection | | | | 40 |
| 318 | Fluorouracil | Intra- | 125 | (F) | Single | Compound I | 1 | h | Intra- | 100 | Single | 7 | 40 |
| 319 | and | peritoneal | 240 | (I) | | | 12 | h | peritoneal | | | | 50 |
| | irinotecan | injection | | | | | | | injection | | | | |
| 320 | Fluorouracil | Intra- | 125 | (F) | Single | Compound III | 0.5 | h | subcutaneous | 100 | Single | 7 | 50 |
| 321 | and | peritoneal | 240 | (I) | | | 24 | h | injection | | | | 20 |
| | irinotecan | injection | | | | | | | | | | | |
| 322 | Fluorouracil | Intra- | 100 | (F) | Single | Compound I | 0.5 | h | Intra- | 300 | Single | 8 | 30 |
| 323 | and | peritoneal | 5 | (O) | | | 4 | h | peritoneal | | | | 30 |
| 324 | oxaliplatin | injection | 100 | (I) | | | 8 | h | injection | | | | 60 |
| 325 | and | | | | | | 12 | h | | | | | 50 |
| 326 | irinotecan | | | | | | 24 | h | | | | | 40 |
| 327 | | | | | | | 48 | h | | | | | 0 |
| 328 | Fluorouracil | Intra- | 100 | (F) | Single | Compound I | 0.5 | h | Intra- | 100 | Single | 8 | 20 |
| 329 | and | venous | 5 | (O) | | | 1 | h | venous | | | | 30 |
| 330 | oxaliplatin | injection | 100 | (I) | | | 2 | h | injection | | | | 30 |
| 331 | and | | 100 | (L) | | | 3 | h | | | | | 50 |
| 332 | irinotecan | | | | | | 4 | h | | | | | 50 |
| 333 | and | | | | | | 6 | h | | | | | 40 |
| 334 | tetrahydro- | | | | | | 8 | h | | | | | 60 |
| 335 | folic acid | | | | | | 10 | h | | | | | 60 |
| 336 | | | | | | | 12 | h | | | | | 60 |
| 337 | | | | | | | 14 | h | | | | | 40 |
| 338 | | | | | | | 16 | h | | | | | 40 |
| 339 | | | | | | | 18 | h | | | | | 40 |
| 340 | | | | | | | 20 | h | | | | | 30 |
| 341 | | | | | | | 24 | h | | | | | 30 |
| 342 | | | | | | | 48 | h | | | | | 0 |
| 343 | Fluorouracil | Intra- | 100 | (F) | Single | Compound I | 0.5 | h | Intra- | 150 | Single | 8 | 30 |
| 344 | and | venous | 5 | (O) | | hydrochloride | 12 | h | peritoneal | | | | 70 |
| | oxaliplatin | injection | 100 | (I) | | | | | injection | | | | |
| | and | | 100 | (L) | | | | | | | | | |
| | irinotecan | | | | | | | | | | | | |
| | and | | | | | | | | | | | | |
| | tetrahydro- | | | | | | | | | | | | |
| | folic acid | | | | | | | | | | | | |
| 345 | Fluorouracil | Intra- | 100 | (F) | Single | Compound I | 6 | h | Oral | 100 | Single | 7 | 60 |
| 346 | and | peritoneal | 5 | (O) | | | 24 | h | adminis- | | | | 40 |
| | oxaliplatin | injection | 100 | (I) | | | | | tration | | | | |
| | and | | 100 | (L) | | | | | | | | | |
| | irinotecan | | | | | | | | | | | | |
| | and | | | | | | | | | | | | |
| | tetrahydro- | | | | | | | | | | | | |
| | folic acid | | | | | | | | | | | | |
| 347 | Fluorouracil | Intra- | 100 | (F) | Single | Compound II | 0.5 | h | Intra- | 100 | Single | 7 | 70 |
| 348 | and | peritoneal | 5 | (O) | | | 2 | h | venous | | | | 50 |
| | oxaliplatin | injection | 100 | (I) | | | | | injection | | | | |
| | and | | 100 | (L) | | | | | | | | | |
| | irinotecan | | | | | | | | | | | | |
| | and | | | | | | | | | | | | |
| | tetrahydro- | | | | | | | | | | | | |
| | folic acid | | | | | | | | | | | | |
| 349 | Fluorouracil | Intra- | 100 | (F) | Single | Compound III | 6 | h | Oral | 150 | Single | 7 | 30 |
| 350 | and | peritoneal | 5 | (O) | | | 12 | h | adminis- | | | | 30 |
| | oxaliplatin | injection | 100 | (I) | | | | | tration | | | | |
| | and | | 100 | (L) | | | | | | | | | |
| | irinotecan | | | | | | | | | | | | |
| | and | | | | | | | | | | | | |
| | tetrahydro- | | | | | | | | | | | | |
| | folic acid | | | | | | | | | | | | |
| 351 | Fluorouracil | Intra- | 100 | (F) | Single | Compound IV | 4 | h | Oral | 100 | Single | 7 | 60 |
| 352 | and | peritoneal | 5 | (O) | | | 24 | h | adminis- | | | | 30 |
| | oxaliplatin | injection | 100 | (I) | | | | | tration | | | | |
| | and | | 100 | (L) | | | | | | | | | |
| | irinotecan | | | | | | | | | | | | |
| | and | | | | | | | | | | | | |
| | tetrahydro- | | | | | | | | | | | | |
| | folic acid | | | | | | | | | | | | |
| 353 | Fluorouracil | Intra- | 100 | (F) | Single | Compound IV | 0.5 | h | Intra- | 100 | Single | 7 | 20 |
| 354 | and | peritoneal | 5 | (O) | | hydrochloride | 12 | h | peritoneal | | | | 60 |
| | oxaliplatin | injection | 100 | (I) | | | | | injection | | | | |
| | and | | 100 | (L) | | | | | | | | | |
| | irinotecan | | | | | | | | | | | | |
| | and | | | | | | | | | | | | |
| | tetrahydro- | | | | | | | | | | | | |
| | folic acid | | | | | | | | | | | | |
| 355 | Fluorouracil | Oral | 100 | (F) | Single | Compound I | 0.5 | h | Sub- | 100 | Single | 7 | 50 |
| 356 | and | adminis- | 5 | (O) | | | 4 | h | cutaneous | | | | 50 |
| | oxaliplatin | tration | 200 | (I) | | | | | injection | | | | |
| | and | | 100 | (L) | | | | | | | | | |
| | irinotecan | | | | | | | | | | | | |
| | and | | | | | | | | | | | | |
| | tetrahydro- | | | | | | | | | | | | |
| | folic acid | | | | | | | | | | | | |
| 357 | Topotecan | Intra- | 10 | | Single | Compound IV | 24 | h | Oral | 100 | Single | 14 | 30 |
| | | peritoneal | | | | hydrochloride | | | adminis- | | | | |
| | | injection | | | | | | | tration | | | | |
| 358 | Topotecan | Intra- | 20 | | Single | Compound IV | 12 | h | Oral | 100 | Single | 14 | 70 |
| | | venous | | | | hydrochloride | | | adminis- | | | | |
| | | injection | | | | | | | tration | | | | |
| 359 | Gemcitabine | Intra- | 120 | | Single | Compound II | 12 | h | Oral | 100 | Single | 8 | 40 |
| | (GEM) | peritoneal | | | | | | | adminis- | | | | |
| | | injection | | | | | | | tration | | | | |
| 360 | Gemcitabine | Intra- | 120 | | Single | Compound I | 24 | h | Oral | 100 | Single | 8 | 30 |
| | (GEM) | venous | | | | | | | adminis- | | | | |
| | | injection | | | | | | | tration | | | | |
| 361 | Etoposide | Intra- | 60 | (E) | Single | Compound II | 2 | h | Oral | 100 | Single | 10 | 60 |
| | and | peritoneal | 5 | (C) | | | | | adminis- | | | | |
| | carboplatin | injection | | | | | | | tration | | | | |
| 362 | Etoposide | Intra- | 60 | (E) | Single | Compound III | 0.5 | h | Oral | 100 | Single | 10 | 50 |
| | and | peritoneal | 5 | (C) | | | | | adminis- | | | | |
| | carboplatin | injection | | | | | | | tration | | | | |
| 363 | Gemcitabine | Intra- | 120 | | Single | Compound IV | 4 | h | Oral | 100 | Single | 8 | 60 |
| | and | peritoneal | | | | | | | adminis- | | | | |
| | carboplatin | injection | | | | | | | tration | | | | |
| 364 | Gemcitabine | Intra- | 120 | (G) | Single | Compound II | 24 | h | Oral | 100 | Single | 8 | 30 |
| | and | venous | 5 | (C) | | | | | adminis- | | | | |
| | carboplatin | injection | | | | | | | tration | | | | |

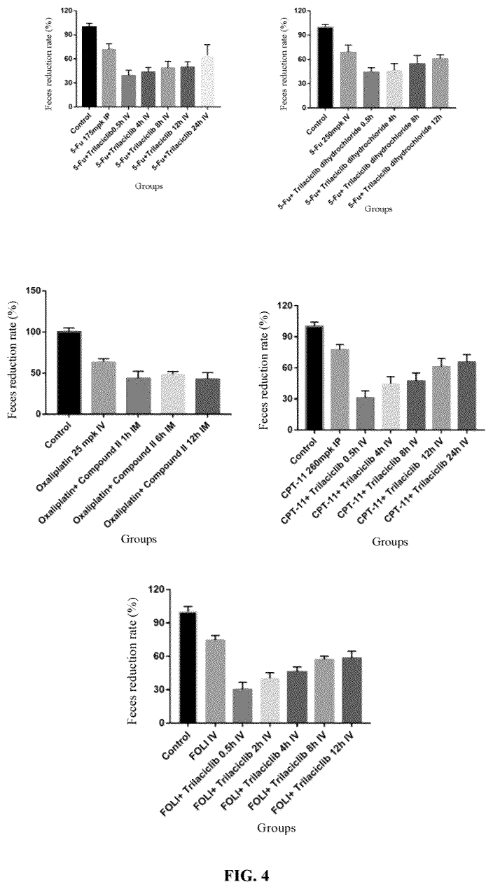

FIG. 3 shows typical photographs showing the feces amount within 3 hours in the control group, the chemotherapeutic agent group, and the group of the compound as shown in Formula I in Table 4. FIG. 4 shows partial results of feces reduction rate in the control group, the chemotherapeutic agent group, and the group of the compound as shown in Formula I in Table 4.

It can be seen from the results in Table 4 and FIG. 4 that, the feces reduction rates in the groups of compound I, II, IV and III, or their hydrochlorides were reduced to different degrees compared with that in the chemotherapeutic agent group. Therefore, the administration of the compound I, II, IV and III, or their hydrochlorides before the chemotherapeutic agent can effectively relieve constipation caused by the chemotherapeutic agent.

### Examples 365-384: Relieving Effect of the Compound as Shown in Formula I on Diarrhea Caused by the Continuous Administration of a Chemotherapeutic Agent

Balb/c mice were fed for 1 week, and then divided into groups. The experiment was divided into a control group, a chemotherapeutic agent group, and a group of the compound as shown in Formula I, each of which comprised 10 mice, to perform the administration experiment, for which the administration dosage, mode, time and frequency were shown in Table 5. The control group: being injected or intragastrically administered with the same solvent as that used in the group of the compound as shown in Formula I (the administration mode and time were shown in Table 5), and then being injected or intragastrically administered with the same solvent as that used in the chemotherapeutic agent group (the administration mode was shown in Table 5); the chemotherapeutic agent group: being injected or intragastrically administered with the same solvent as that used in the group of the compound as shown in Formula I (the administration mode and time were shown in Table 5), and then being injected or intragastrically administered with the chemotherapeutic agent (the type, administration mode, time and dosage were shown in Table 5); the group of the compound as shown in Formula I: being injected or intragastrically administered with the compound as shown in Formula I (the administration mode and time were shown in Table 5), and then being administered with the chemotherapeutic agent (the type, administration mode, time and dosage were shown in Table 5).

### Diarrhea Observation

The scoring of diarrhea can refer to the method of Akinobu Kurita (Cancer Chemother Pharmacol 2000; 46:211-20.), Grade 0): normal feces; Grade 1: mild diarrhea, the stools is slightly wet and soft; Grade 2: moderate diarrhea, loose stools and mild perianal contamination; Grade 3: severe diarrhea, watery stools and accompanied by severe perianal staining (see FIG. 1). It is regarded as effective remission when the diarrhea grading of mice in the group of the compound as shown in Formula I is reduced compared with the average diarrhea grading of mice in the chemotherapeutic agent group.

The diarrhea grading of mice was observed and recorded every day. At the end of the experiment, the number of mice in the group of the compound as shown in Formula I whose diarrhea grading was lower than that of mice in the chemotherapeutic agent group was recorded. Table 5 lists various animal experiment combinations of chemotherapeutic agents and the compound as shown in Formula I, as well as the corresponding experimental results. (Relative remission rate %=the number of mice in the group of the compound as shown in Formula I whose diarrhea has been effectively relieved/the total number of mice in the group of the compound as shown in Formula I*100%. The establishment rate of diarrhea model in the chemotherapeutic agent group was 50%-80%, that is, about 5-8 of 10 mice developed diarrhea during the experiment, and there were cases of individual mice death or not developing diarrhea.)

TABLE 5
Experimental Conditions and Results of Examples 365-384

| | | | | | | Adminis- | | | | | | |
|---|---|---|---|---|---|---|---|---|---|---|---|---|
| | | | | | | tration | | | | | | |
| | | | | | | time | | | | | | |
| | | | | | | (before the | | | | | Experi- | Relative |
| Exam- | Chemo- | Adminis- | | | Compound | chemo- | | Adminis- | | | mental | remission |
| ple | therapeutic | tration | Dosage | | as shown in | therapeutic | | tration | Dosage | | cycle | rate |
| No. | agent | mode | (mg/kg) | Frequency | Formula I | agent) | | mode | (mg/kg) | Frequency | (days) | (%) |
| 365 | Fluorouracil | Intra- | 70 | Once a | Compound I | 2 | h | Intra- | 100 | Once a | 10 | 60 |
| 366 | (5-Fu) | peritoneal | | day, for | | 4 | h | venous | | day, for | | 40 |
| 367 | | injection | | 3 days | | 6 | h | injection | | 3 days | | 30 |
| 368 | | | | | | 8 | h | | | | | 20 |
| 369 | Fluorouracil | Intra- | 70 | Once a | Compound I | 2 | h | Intra- | 100 | Once a | 9 | 50 |
| 370 | (5-Fu) | peritoneal | | day, for | | 4 | h | peritoneal | | day, for | | 30 |
| 371 | | injection | | 5 days | | 6 | h | injection | | 5 days | | 20 |
| 372 | | | | | | 8 | h | | | | | 10 |
| 373 | Fluorouracil | Intra- | 70 | Once a | Compound I | 2 | h | Intra- | 150 | Single | 9 | 60 |
| 374 | (5-Fu) | peritoneal | | day for | hydrochloride | 4 | h | peritoneal | | | | 30 |
| 375 | | injection | | 5 days | | 8 | h | injection | | | | 10 |
| 376 | Fluorouracil | Intra- | 70 | Once a | Compound I | 2 | h | Intra- | 125 | Once a | 10 | 10 |
| 377 | (5-Fu) | peritoneal | | day, for | | 4 | h | peritoneal | | day, for | | |
| 378 | | injection | | 7 days | | 6 | h | injection | | 7 days | | |
| 379 | | | | | | 8 | h | | | | | |
| 380 | Fluorouracil | Intra- | 70 | Once a | Compound IV | 4 | h | Oral | 100 | Once a | 8 | 30 |
| | (5-Fu) | peritoneal | | day, for | | | | adminis- | | day, for | | |
| | | injection | | 3 days | | | | tration | | 3 days | | |
| 381 | Fluorouracil | Intra- | 70 | Once a | Compound II | 0.5 | h | Intra- | 100 | Once a | 8 | 40 |
| | (5-Fu) | venous | | day, for | | | | muscular | | day, for | | |
| | | injection | | 3 days | | | | injection | | 3 days | | |
| 382 | Fluorouracil | Intra- | 70 | Once a | Compound III | 12 | h | sub- | 100 | Once a | 8 | 20 |
| | (5-Fu) | venous | | day, for | | | | cutaneous | | day, for | | |
| | | injection | | 3 days | | | | injection | | 3 days | | |
| 383 | Fluorouracil | Intra- | 70 | Once a | Compound I | 6 | h | Oral | 100 | Once a | 8 | 50 |
| | (5-Fu) | venous | | day for | | | | adminis- | | day, for | | |
| | | injection | | 3 days | | | | tration | | 3 days | | |
| 384 | Fluorouracil | Oral | 50 | Once a | Compound I | 8 | h | Intra- | 100 | Once a | 14 | Ineffective |
| | (5-Fu) | adminis- | | day, for | | | | peritoneal | | day, for | | |
| | | tration | | 14 days | | | | injection | | 14 days | | |

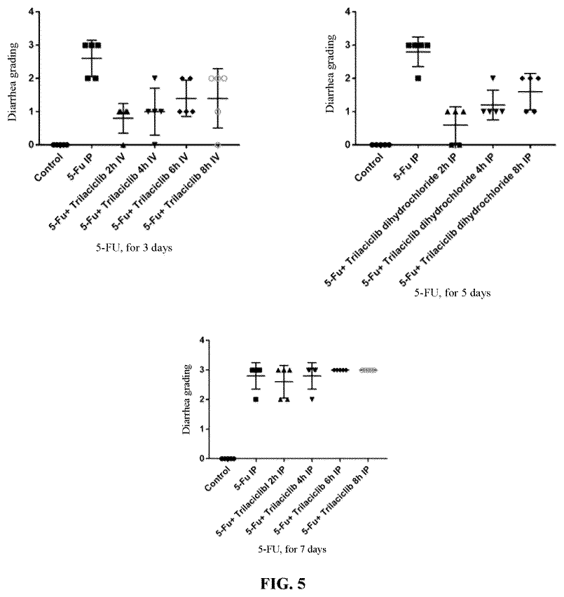

It can be seen from Table 5 and FIG. 5 that: the compound I, II, IV and III, and their hydrochlorides have relieving effects on diarrhea caused by the continuous administration of the chemotherapeutic agent for 7 days or less, and the relieving effects on diarrhea caused by the continuous administration of the chemotherapeutic agent for less than 7 days were significant. Compound I is ineffective to the relieving effect on diarrhea caused by the continuous administration of fluorouracil for 14 days.

### Examples 385-405: Relieving Effect of Oral Administration of the Compound as Shown in Formula I on Constipation Caused by the Continuous Administration of a Chemotherapeutic Agent

Balb/c mice were fed for 1 week, and then divided into groups. The experiment was divided into a control group, a chemotherapeutic agent group, and a group of the compound as shown in Formula I, each of which comprised 10 mice, to perform the administration experiment, for which the administration dosage, mode, time and frequency were shown in Table 6. The control group: being injected or intragastrically administered with the same solvent as that used in the group of the compound as shown in Formula I (the administration mode and time were shown in Table 6), and then being injected or intragastrically administered with the same solvent as that used in the group of the compound as shown in Formula I (the administration mode was shown in Table 6); the chemotherapeutic agent group: being injected or intragastrically administered with the same solvent as that used in the group of the compound as shown in Formula I (the administration mode and time were shown in Table 6), and then being injected or intragastrically administered with the chemotherapeutic agent (the type, administration mode, time and dosage were shown in Table 6); the group of the compound as shown in Formula I: being injected or intragastrically administered with the compound as shown in Formula I (the administration mode and time were shown in Table 6), and then being administered with the chemotherapeutic agent (the type, administration mode, time and dosage were shown in Table 6).

### Constipation Observation

The feces of mice within 3 hours were collected, which were observed for shape and weighed with an electronic balance. The feces reduction rate was calculated with reference to the method of Ji Eun Kim (Lab Anim Res. 2016 December; 32(4): 231-240.). Feces reduction rate (%)=(the control group−the chemotherapeutic agent group or the group of the compound as shown in Formula I)/the control group*100%

It is regarded as effective remission when the feces reduction rate of mice in the group of the compound as shown in Formula I is reduced compared with that in the chemotherapeutic agent group.

The feces amount of mice within 3 hours was observed and recorded every day. At the end of the experiment, the number of mice in the group of the compound as shown in Formula I whose feces reduction rate was lower than that of mice in the chemotherapeutic agent group was recorded. Table 6 lists various animal experiment combinations of chemotherapeutic agents and the compound as shown in Formula I, as well as the corresponding experimental results. (Relative remission rate %=the number of mice in the group of the compound as shown in Formula I which have been effectively relieved/the total number of mice in the group of the compound as shown in Formula I*100%. The establishment rate of constipation model in the chemotherapeutic agent group was 30%-60%, that is, about 3-6 of 10 mice showed reduced feces amount during the experiment, and there were cases of individual mice death or with no changes or an increasement in the feces amount.)

TABLE 6
Experimental Conditions and Results of Examples 385-405

| | | | | | | Adminis- | | | | | | |
|---|---|---|---|---|---|---|---|---|---|---|---|---|
| | | | | | | tration | | | | | | |
| | | | | | | time | | | | | | |
| | | | | | | (before the | | | | | Experi- | Relative |
| Exam- | Chemo- | Adminis- | | | Compound | chemo- | | Adminis- | | | mental | remission |
| ple | therapeutic | tration | Dosage | | as shown in | therapeutic | | tration | Dosage | | cycle | rate |
| No. | agent | mode | (mg/kg) | Frequency | Formula I | agent) | | mode | (mg/kg) | Frequency | (days) | (%) |
| 385 | Fluorouracil | Intra- | 100 | Once a | Compound I | 0.5 | h | Intra- | 100 | Once a | 7 | 70 |
| 386 | (5-Fu) | peritoneal | | day, for | | 4 | h | venous | | day, for | | 50 |
| 387 | | injection | | 3 days | | 6 | h | injection | | 3 days | | 40 |
| 388 | | | | | | 8 | h | | | | | 30 |
| 389 | Fluorouracil | Intra- | 100 | Once a | Compound I | 0.5 | h | Intra- | 100 | Once a | 9 | 60 |
| 390 | (5-Fu) | peritoneal | | day, for | | 12 | h | peritoneal | | day, for | | 30 |
| 391 | | injection | | 5 days | | 20 | h | injection | | 5 days | | 10 |
| 392 | | | | | | 24 | h | | | | | 10 |
| 393 | Fluorouracil | Intra- | 100 | Once a | Compound I | 0.5 | h | Intra- | 150 | Once a | 9 | 70 |
| 394 | (5-Fu) | peritoneal | | day for | hydrochloride | 4 | h | peritoneal | | day, for | | 50 |
| 395 | | injection | | 5 days | | 12 | h | injection | | 5 days | | 30 |
| 396 | Fluorouracil | Intra- | 100 | Once a | Compound I | 4 | h | Oral | 100 | Once a | 7 | 40 |
| | (5-Fu) | venous | | day, for | | | | adminis- | | day, for | | |
| | | injection | | 3 days | | | | tration | | 3 days | | |
| 397 | Fluorouracil | Intra- | 100 | Once a | Compound II | 0.5 | h | Intra- | 100 | Once a | 7 | 20 |
| | (5-Fu) | peritoneal | | day, for | | | | peritoneal | | day, for | | |
| | | injection | | 3 days | | | | injection | | 3 days | | |
| 398 | Fluorouracil | Intra- | 100 | Once a | Compound III | 2 | h | Intra- | 100 | Once a | 7 | 40 |
| | (5-Fu) | peritoneal | | day, for | | | | venous | | day, for | | |
| | | injection | | 3 days | | | | injection | | 3 days | | |
| 399 | Fluorouracil | Intra- | 100 | Once a | Compound IV | 4 | h | Oral | 100 | Once a | 7 | 50 |
| | (5-Fu) | peritoneal | | day for | | | | adminis- | | day, for | | |
| | | injection | | 3 days | | | | tration | | 3 days | | |
| 400 | Fluorouracil | Intra- | 100 | Once a | Compound IV | 2 | h | Intra- | Once a | Single | 7 | 50 |
| 401 | (5-Fu) | peritoneal | | day, for | hydrochloride | 12 | h | peritoneal | day, for | | | 30 |
| | | injection | | 3 days | | | | injection | 3 days | | | |
| 402 | Fluorouracil | Intra- | 100 | Once a | Compound I | 2 | h | Intra- | 125 | Once a | 10 | 10 |
| 403 | (5-Fu) | peritoneal | | day for | | 4 | h | peritoneal | | day, for | | 10 |
| 404 | | injection | | 7 days | | 6 | h | injection | | 7 days | | 10 |
| 405 | | | | | | 8 | h | | | | | 0 |

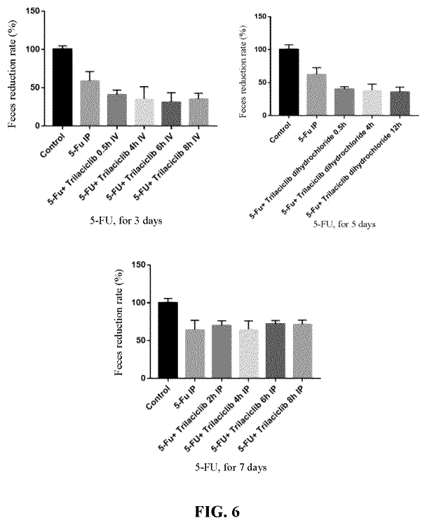

It can be seen from Table 6 and FIG. 6 that: the compound I, II, IV and III, and their hydrochlorides have relieving effects on constipation caused by the continuous administration of the chemotherapeutic agent for 7 days or less, and especially have significant relieving effects on constipation caused by the continuous administration of fluorouracil for less than 7 days.

1. A method for preventing, relieving, and/or treating one or more gastrointestinal side effects associated with chemotherapy in a subject in need thereof, the method comprising:
administering to the subject a compound of formula (I), optionally as a pharmaceutically acceptable salt;
wherein 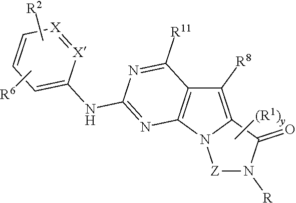
(I),
Z is —(CH₂)_{X}, with X being 1, 2, 3, or 4, or —O—(CH₂)_{Z}—, with z being 2, 3 or 4;
X and X′ are each independently C of N;
R, R⁸, and R¹¹ are each independently H, C₁-C₃ alkyl, C₁-C₃ haloalkyl, cycloalkyl optionally comprising N, O, and/or S, -(alkylene)ₘ-C₃-C₈ cycloalkyl, -(alkylene)ₘ-aryl, -(alkylene)ₘ-heterocyclyl, -(alkylene)ₘ-heteroaryl, -(alkylene)ₘ-NR³R⁴, -(alkylene)ₘ-C(O)—NR³R⁴, -(alkylene)ₘ-O—R⁵, -(alkylene)ₘ-S(O)ₙ—R⁵, or -(alkylene)ₘ-S(O)ₙ—NR³R⁴, any of which is optionally independently substituted with one or more R groups as allowed by valance, and wherein two R^{x} groups bound to the same or adjacent atoms optionally combine to form a ring, and wherein m is 0 or 1, and n is 0, 1, or 2,
R¹ is independently aryl, alkyl, cycloalkyl, or haloalkyl, each of which optionally comprises O or N heteroatom(s) in place of a carbon in the chain, and two R¹s on adjacent ring atoms or on the same ring atom together with the ring atom(s) to which they are attached optionally form a 3-8-membered cycle;
y is 0, 1, 2, 3, or 4,
R² is -(alkylene)ₘ-heterocyclyl, -(alkylene)ₘ-heteroaryl, -(alkylene)ₘ-NR³R⁴, -(alkylene)ₘ-C(O)—NR³R⁴, -(alkylene)ₘ-C(O)—O-alkyl; -(alkylene)ₘ-O—R⁵, -(alkylene)ₘ-S(O)ₙ—R⁵ or -(alkylene)ₘ-S(O)ₙ—NR³R⁴, any of which is optionally independently substituted with one or more R^{x} groups as allowed by valance, and wherein two R^{x} groups bound to the same or adjacent atoms optionally combine to form a ring, and wherein m is 0 or 1, and n is 0, 1, or 2;
R³ and R⁴ at each occurrence are independently H, alkyl, cycloalkyl, heterocyclyl, aryl, heteroaryl, cycloalkylalkyl, heterocycloalkyl, arylalkyl, or heteroarylalkyl, any of which is optionally independently substituted with one or more R^{x} groups as allowed by valance, and wherein two R^{x} groups bound to the same or adjacent atoms optionally combine to form a ring; or R³ and R⁴ together with the nitrogen atom to which they are attached may combine to form a heterocyclic ring optionally independently substituted with one or more R^{x} groups as allowed by valance, and wherein two R^{x} groups bound to the same or adjacent atoms optionally combine to form a ring;
R⁵ and R⁵* at each occurrence are H, alkyl, alkenyl, alkynyl, cycloalkyl, heterocyclyl, aryl, heteroaryl, cycloalkylalkyl, heterocycloalkyl, arylalkyl, or heteroarylalkyl, any of which is optionally independently substituted with one or more R^{x} groups as allowed by valance;
R^{x} at each occurrence is independently halo, cyano, nitro, oxo, alkyl, haloalkyl, alkenyl, alkynyl, cycloalkyl, cycloalkenyl, heterocyclyl, aryl, heteroaryl, arylalkyl, heteroarylalkyl, cycloalkylalkyl, heterocycloalkyl, -(alkylene)ₘ-OR⁵, -(alkylene)ₘ-O-alkylene-OR⁵, -(alkylene)ₘ-S(O)ₙ—R⁵, -(alkylene)ₘ-NR³R⁴, -(alkylene)ₘ-CN, -(alkylene)ₘ-C(O)—R⁵, -(alkylenc)ₘ-C(S)—R⁵, -(alkylene)ₘ-C(O)—OR⁵, -(alkylenc)ₘ-O—C(O)—R⁵, -(alkylene)ₘ-C(S)—OR⁵, -(alkylene)ₘ-C(O)-(alkylene)ₘ-NR³R⁴, -(alkylene)ₘ-C(S)—NR³R⁴, -(alkylene)ₘ-N(R³)—C(O)—NR³R⁴, -(alkylene)ₘ-N(R³)—C(S)—NR³R⁴, -(alkylene)ₘ-N(R³)—C(O)—R⁵, -(alkylene)ₘ-N(R³)—C(S)—R⁵, -(alkylene)ₘ-O—C(O)—NR³R⁴, -(alkylene)ₘ-O—C(S)—NR³R⁴, -(alkylene)ₘ-SO₂—NR³R⁴, -(alkylene)ₘ-N(R³)—SO₂—R⁵, -(alkylene)ₘ-N(R³)—SO₂—NR³R⁴, -(alkylene)ₘ-N(R³)—C(O)—OR⁵, -(alkylene), —N(R³)—C(S)—OR⁵, or -(alkylene)ₘ-N(R³)—SO₂—R⁵; the alkyl, haloalkyl, alkenyl, alkynyl, cycloalkyl, cycloalkenyl, heterocyclyl, aryl, heteroaryl, arylalkyl, heteroarylalkyl, cycloalkylalkyl, and heterocycloalkyl optionally being further independently substituted with one or more -(alkylene)ₘ-CN, -(alkylene)ₘ-OR⁵*, -(alkylene)ₘ-S(O)ₙ—R⁵*, -(alkylene)ₘ-NR³*R⁴*, -(alkylene)ₘ—C(O)—R⁵*, -(alkylene)ₘ-C(═S)R⁵*, -(alkylene)ₘ-C(═O)OR⁵*, -(alkylene)ₘ-(C(═O)R⁵*, -(alkylene)ₘ-C(S)—OR⁵*, -(alkylene)ₘ-C(O)—NR³*R⁴*, -(alkylene)ₘ-C(S)—NR³*R⁴*, -(alkylene)ₘ-N(R³*)—C(O))—NR³*R⁴*, -(alkylene)ₘ-N(R³*)—C(S)—NR³*R⁴*, -(alkylene)ₘ-N(R³*)—C(O)—R⁵*, -(alkylene)ₘ-N(R³*)—C(S)—R⁵*, -(alkylene)ₘ-O—C(O)—NR³*R⁴*, -(alkylene)ₘ-O—C(S)—NR³*R⁴*, -(alkylene)ₘ-SO₂—NR³*R⁴*, -(alkylene)ₘ-N(R³*)—SO₂—R⁵*, -(alkylene)ₘ-N(R³*)—SO₂—NR³*R⁴*, -(alkylene)ₘ-N(R³*)—C(O)—OR⁵*, -(alkylene)ₘ-N(R³*)—C(S)—OR⁵*, or -(alkylene)ₘ-N(R³*)—SO₂—R⁵*,
n is 0, 1, or 2,
m is 0 or 1,
R³* and R⁴* at each occurrence are independently H, alkyl, alkenyl, alkynyl, cycloalkyl, heterocyclyl, aryl, heteroaryl, cycloalkylalkyl, heterocycloalkyl, arylalkyl or heteroarylalkyl, any of which is optionally independently substituted with one or more R^{x} groups as allowed by valance; or R³* and R⁴* together with the nitrogen atom to which they are attached combine to form a heterocyclic ring optionally independently substituted with one or more R^{x} groups as allowed by valance; and
R⁶ is H, lower alkyl, -(alkylene)ₘ-heterocyclyl, -(alkylene)ₘ-heteroaryl, -(alkylene)ₘ-NR³R⁴, -(alkylene)ₘ-C(O)—NR³R⁴; -(alkylene)ₘ-O—R⁵, -(alkylene)ₘ-S(O)ₙ—R⁵, or -(alkylene)ₘ-S(O)ₙ—NR³R⁴, any of which may be is optionally independently substituted with one or more R^{x} groups as allowed by valance, and wherein two R groups bound to the same or adjacent atoms optionally combine to form a ring.

2-5. (canceled)

6. The method of claim 1, wherein the compound has formula (Ia): 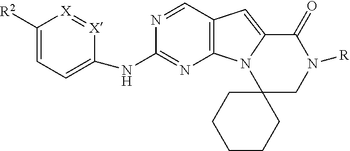
(Ia).

7-9. (canceled)

10. The method of claim 1, wherein R² is -(alkylene)ₘ-heterocyclyl, -(alkylene)ₘ-NR³R⁴, -(alkylene)ₘ-C(O)—NR³R⁴, -(alkylene)ₘ-C(O)—O-alkyl, or -(alkylene)ₘ-OR⁵, any of which is optionally independently substituted with one or more R^{x} groups as allowed by valance,
wherein two R^{x} groups bound to the same or adjacent atoms optionally combine to form a ring, and
wherein m is 0 or 1.

11. The method of claim 1, wherein R² is -(alkylene)ₘ-heterocyclyl, -(alkylene)ₘ-NR³R⁴, -(alkylene)ₘ-C(O)—NR³R⁴, -(alkylene)ₘ-C(O)—O-alkyl, or -(alkylene)ₘ-OR³, which is not further substituted.

12-13. (canceled)

14. The method of claim 1, wherein
R² is 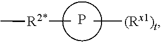
R^{2*} is a bond, alkylene, -(alkylene)ₘ-O-(alkylene)ₘ-, -(alkylene)ₘ-C(O)-(alkylene)ₘ-, -(alkylene)ₘ-S(O)₂-(alkylene)ₘ-, or -(alkylene)ₘ-NH-(alkylene)ₘ-, each m independently being 0 or 1;
P is a 4- to 8-membered mono- or bicyclic saturated heterocyclyl group,
each R^{x1} is independently -(alkylene)ₘ-(C(O))ₘ-(alkylene)ₘ-(N(R^{N}))ₘ-(alkyl)ₘ, each m being independently 0 or 1 provided at least one m is 1, —(C(O))—O-alkyl, -(alkylene)ₘ-cycloalkyl m being 0 or 1 —N(R^{N})-cycloalkyl, —C(O)-cycloalkyl, -(alkylene)ₘ-heterocyclyl, m being 0 or 1, or —N(R^{N})-heterocyclyl, —C(O)-heterocyclyl —S(O)₂-(alkylene)ₘ, m being 1 or 2, R^{N} being H, C₁ to C₄ alkyl, or C₁ to C₆ heteroalkyl, and two R^{x1} optionally form a ring, together with atoms to which they attach on P, which is optionally the same atom, form a ring, and
t is 0, 1, or 2.

15.-17. (canceled)

18. The method of claim 1, wherein
R² is 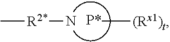
and
P* is a 4- to 8-membered mono- or bicyclic saturated heterocyclyl group.

19. The method of claim 1, wherein
R² is

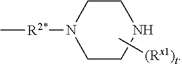

20. The method of claim 1, wherein
R² is

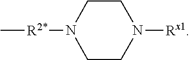

21. The method of claim 1, wherein
R² is 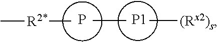
wherein
R²* is a bond, alkylene, -(alkylene)ₘ-O-(alkylene)ₘ-, -(alkylene)ₘ-C(O))-(alkylene)ₘ-, -(alkylene)ₘ-S(O)₂-(alkylene)ₘ, or -(alkylene)ₘ-NH-(alkylene)ₘ-, each m being independently 0 or 1;
P is a 4- to 8-membered mono- or bicyclic saturated heterocyclyl group,
P1 is a 4- to 6-membered monocyclic saturated heterocyclyl group,
each R^{x2} is independently hydrogen or alkyl, and
s is 0, 1, or 2.

22. The method of claim 1, wherein said R² is

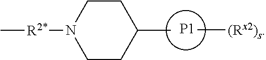

23-27. (canceled)

28. The method of claim 1, wherein the compound of formula (I):

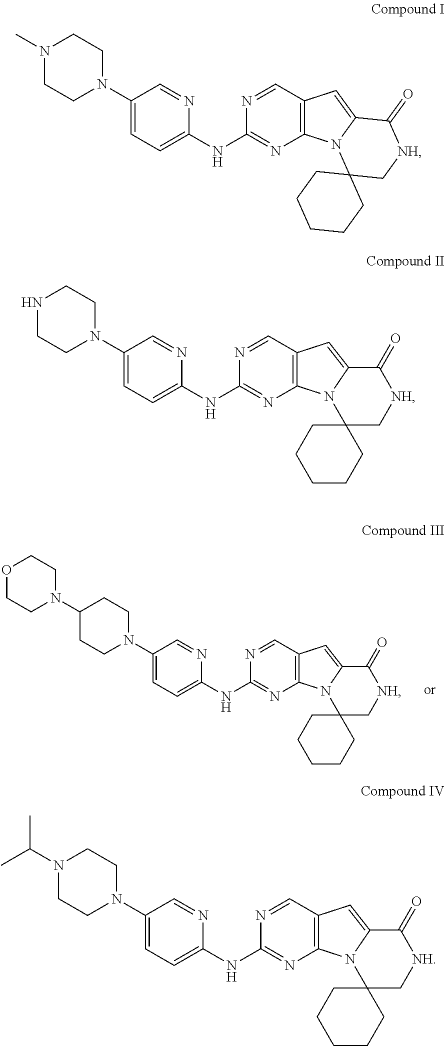 [SMILES: CN1CCN(c2ccc(Nc3ncc4cc5n(c4n3)C3(CCCCC3)CNC5=O)nc2)CC1 ; O=C1NCC2(CCCCC2)n2c1cc1cnc(Nc3ccc(N4CCNCC4)cn3)nc12 ; O=C1NCC2(CCCCC2)n2c1cc1cnc(Nc3ccc(N4CCC(N5CCOCC5)CC4)cn3)nc12 ; "or" ; CC(C)N1CCN(c2ccc(Nc3ncc4cc5n(c4n3)C3(CCCCC3)CNC5=O)nc2)CC1]

29-30. (canceled)

31. The method of claim 1, wherein the chemotherapy comprises administering a chemotherapeutic agent.

32. The method of claim 31, wherein the chemotherapeutic agent is a cytotoxic agent.

33-34. (canceled)

35. The method of claim 31, wherein the chemotherapeutic agent comprises fluorouracil, oxaliplatin, irinotecan, topotecan, etoposide, cisplatin, docetaxel, gemcitabine, carboplatin, methotrexate, doxorubicin, cytarabine, vinorelbine, and/or capecitabine.

36. (canceled)

37. The method of claim 31, wherein the one or more gastrointestinal side effects comprise a gastrointestinal side effect caused by the chemotherapeutic agent.

38. The method of claim 31, wherein the one or more gastrointestinal side effects occur or deteriorate after administration of the chemotherapeutic agent.

39. (canceled)

40. The method of claim 31, wherein the one or more gastrointestinal side effects comprise gastric mucosal injury diseases and/or intestinal mucosal injury diseases.

41. The method of claim 1, wherein the one or more gastrointestinal side effects comprise diarrhea, abdominal pain, nausea, vomiting, mucositis, loss of appetite, gastric ulcer, gastritis, constipation, enteritis, intestinal perforation, intestinal bleeding, ulcer, and/or intestinal necrosis.

42-45. (canceled)

46. The method of claim 1, wherein the compound of formula (I) is administered at least 0.5 hours before the administration of the chemotherapy.

47. (canceled)

48. The method of claim 1, wherein the compound of formula (I) is prepared for oral administration.

49-55. (canceled)
